(19) United States
(12) Patent Application Publication
(10) Pub. No.: US 2009/0325957 A1
(43) Pub. Date: Dec. 31, 2009

(54) MORPHOLINO PYRIMIDINE DERIVATIVES USEFUL IN THE TREATMENT OF PROLIFERATIVE DISORDERS

(75) Inventor: Maurice Raymond Verschoyle Finlay, Cheshire (GB)

Correspondence Address: MORGAN LEWIS & BOCKIUS LLP, 1111 PENNSYLVANIA AVENUE NW, WASHINGTON, DC, 20004, US

(73) Assignee: ASTRAZENECA AB, Sodertalje (SE)

(21) Appl. No.: 12/438,477

(22) PCT Filed: Aug. 22, 2007
(86) PCT No.: PCT/GB2007/003211

§ 371 Date: Feb. 23, 2009

Related U.S. Application Data
(60) Provisional application No. 60/948,537, filed on Jul. 9, 2007.

(30) Foreign Application Priority Data
Aug. 24, 2006 (GB) 0616747.2

(51) Int. Cl. A61K 31/5377; C07D 413/04

(52) U.S. Cl. 514/235.8; 544/122

(57) ABSTRACT

A compound of formula (I)

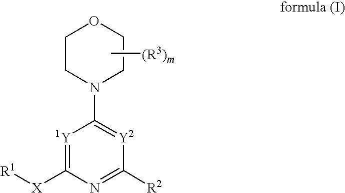

or a pharmaceutically acceptable salt thereof, processes for their preparation, pharmaceutical compositions containing them and their use in therapy, for example in the treatment of proliferative disease such as cancer and particularly in disease mediated by an mTOR kinase and/or one or more PI3K enzyme.

The present invention relates to molpholino pyrimidine derivatives, processes for their preparation, pharmaceutical compositions containing them and their use in therapy, for example in the treatment of proliferative disease such as cancer and particularly in disease mediated by an mTOR kinase and/or one or more PI3K enzyme.

It is now well understood that deregulation of oncogenes and tumour-suppressor genes contributes to the formation of malignant tumours, for example by way of increased cell proliferation or increased cell survival. It is also known that signalling pathways mediated by the PI3K/mTOR families have a central role in a number of cell processes including proliferation and survival, and deregulation of these pathways is a causative factor in a wide spectrum of human cancers and other diseases.

The mammalian target of the macrolide antibiotic Rapamycin (sirolimus) is the enzyme mTOR. This enzymes belongs to the phosphatidylinositol (PI) kinase-related kinase (PIKK) family of protein kinases, which also includes ATM, ATR, DNA-PK and hSMG-1. mTOR, like other PIKK family members, does not possess detectable lipid kinase activity, but instead functions as a serine/threonine kinase. Much of the knowledge of mTOR signalling is based upon the use of Rapamycin. Rapamycin first binds to the 12 kDa immunophilin FK506-binding protein (FKBP12) and this complex inhibits mTOR signalling (Tee and Blenis, Seminars in Cell and Developmental Biology, 2005, 16, 29-37). The mTOR protein consists of a catalytic kinase domain, an FKBP12-Rapamycin binding (FRB) domain, a putative repressor domain near the C-terminus and up to 20 tandemly-repeated HEAT motifs at the N-terminus, as well as FRAP-ATM-TRRAP (FAT) and FAT C-terminus domain (Huang and Houghton, Current Opinion in Pharmacology, 2003, 3, 371-377).

mTOR kinase is a key regulator of cell growth and has been shown to regulate a wide range of cellular functions including translation, transcription, m-RNA turnover, protein stability, actin cytoskeleton reorganisation and autophagy (Jacinto and Hall, Nature Reviews Molecular and Cell Biology, 2005, 4, 117-126). mTOR kinase integrates signals from growth factors (such as insulin or insulin-like growth factor) and nutrients (such as amino acids and glucose) to regulate cell growth. mTOR kinase is activated by growth factors through the PI3K-Akt pathway. The most well characterised function of mTOR kinase in mammalian cells is regulation of translation through two pathways, namely activation of ribosomal S6K1 to enhance translation of mRNAs that bear a 5′-terminal oligopyrimidine tract (TOP) and suppression of 4E-BP1 to allow CAP-dependent mRNA translation.

Generally, investigators have explored the physiological and pathological roles of mTOR using inhibition with Rapamycin and related Rapamycin analogues based on their specificity for mTOR as an intracellular target. However, recent data suggests that Rapamycin displays variable inhibitory actions on mTOR signalling functions and suggest that direct inhibition of the mTOR kinase domain may display substantially broader anti-cancer activities than that achieved by Rapamycin (Edinger et al., Cancer Research, 2003, 63, 8451-8460). For this reason, potent and selective inhibitors of mTOR kinase activity would be useful to allow a more complete understanding of mTOR kinase function and to provide useful therapeutic agents.

There is now considerable evidence indicating that the pathways upstream of mTOR, such as the PI3K pathway, are frequently activated in cancer (Vivanco and Sawyers, Nature Reviews Cancer, 2002, 2, 489-501; Bjornsti and Houghton, Nature Reviews Cancer, 2004, 4, 335-348; Inoki et al., Nature Genetics, 2005, 37, 19-24). For example, components of the PI3K pathway that are mutated in different human tumours include activating mutations of growth factor receptors and the amplification and/or overexpression of PI3K and Akt.

In addition there is evidence that endothelial cell proliferation may also be dependent upon mTOR signalling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signalling is believed to partially control VEGF synthesis through effects on the expression of hypoxia-inducible factor-1α (HIF-1α) (Hudson et al., Molecular and Cellular Biology, 2002, 22, 7004-7014). Therefore, tumour angiogenesis may depend on mTOR kinase signalling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of cancer comprising solid tumours such as carcinomas and sarcomas and the leukaemias and lymphoid malignancies. In particular, inhibitors of mTOR kinase should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

In addition to tumourigenesis, there is evidence that mTOR kinase plays a role in an array of hamartoma syndromes. Recent studies have shown that the tumour suppressor proteins such as TSC1, TSC2, PTEN and LKB1 tightly control mTOR kinase signalling. Loss of these tumour suppressor proteins leads to a range of hamartoma conditions as a result of elevated mTOR kinase signalling (Tee and Blenis, Seminars in Cell and Developmental Biology, 2005, 16, 29-37). Syndromes with an established molecular link to dysregulation of mTOR kinase include Peutz-Jeghers syndrome (PJS), Cowden disease, Bannayan-Riley-Ruvalcaba syndrome (BRRS), Proteus syndrome, Lhermitte-Duclos is disease and Tuberous Sclerosis (TSC) (Inoki et al., Nature Genetics, 2005, 37, 19-24). Patients with these syndromes characteristically develop benign hamartomatous tumours in multiple organs.

Recent studies have revealed a role for mTOR kinase in other diseases (Easton & Houghton, Expert Opinion on Therapeutic Targets, 2004, 8, 551-564). Rapamycin has been demonstrated to be a potent immunosuppressant by inhibiting antigen-induced proliferation of T cells, B cells and antibody production (Sehgal, Transplantation Proceedings, 2003, 35, 7S-14S) and thus mTOR kinase inhibitors may also be useful immunosuppressives. Inhibition of the kinase activity of mTOR may also be useful in the prevention of restenosis, that is the control of undesired proliferation of normal cells in the vasculature in response to the introduction of stents in the treatment of vasculature disease (Morice et al., New England Journal of Medicine, 2002, 346, 1773-1780). Furthermore, the Rapamycin analogue, everolimus, can reduce the severity and incidence of cardiac allograft vasculopathy (Eisen et al., New England Journal of Medicine, 2003, 349, 847-858). Elevated mTOR kinase activity has been associated with cardiac hypertrophy, which is of clinical importance as a major risk factor for heart failure and is a consequence of increased cellular size of cardiomyocytes (Tee & Blenis, Seminars in Cell and Developmental Biology, 2005, 16, 29-37). Thus mTOR kinase inhibitors are expected to be of value in the prevention and treatment of a wide variety of diseases in addition to cancer.

It is also believed that a number of these morpholino pyrimidine derivatives may have inhibitory activity against the phosphatidylinositol (PI) 3-kinases family of kinases.

Phosphatidylinositol (PI) 3-kinases (PI3Ks) are ubiquitous lipid kinases that function both as signal transducers downstream of cell-surface receptors and in constitutive intracellular membrane and protein trafficking pathways. All PI3Ks are dual-specificity enzymes with a lipid kinase activity that phosphorylates phosphoinositides at the 3-hydroxy position, and a less well characterised protein kinase activity. The lipid products of PI3K-catalysed reactions comprising phosphatidylinositol 3,4,5-trisphosphate [PI(3,4,5)P₃], phosphatidylinositol 3,4-bisphosphate [PI(3,4)P₂] and phosphatidylinositol 3-monophosphate [PI(3)P] constitute second messengers in a variety of signal transduction pathways, including those essential to cell proliferation, adhesion, survival, cytoskeletal rearrangement and vesicle trafficking. PI(3)P is constitutively present in all cells and its levels do not change dramatically following agonist stimulation. Conversely, PI(3,4)P₂ and PI(3,4,5)P₃ are nominally absent in most cells but they rapidly accumulate on agonist stimulation.

The downstream effects of PI3K-produced 3-phosphoinositide second messengers are mediated by target molecules containing 3-phosphoinositide binding domains such as the pleckstrin homology (PH) domain and the recently identified FYVE and phox domains.

Well-characterised protein targets for PI3K include PDK1 and protein kinase B (PKB). In addition, tyrosine kinases like Btk and Itk are dependent on PI3K activity.

The PI3K family of lipid kinases can be classified into three groups according to their physiological substrate specificity (Vanhaesebroeck et al., Trends in Biol. Sci., 1997, 22, 267). Class III PI3K enzymes phosphorylate PI alone. In contrast, Class II PI3K enzymes phosphorylate both PI and PI 4-phosphate [PI(4)P]. Class I PI3K enzymes phosphorylate PI, PI(4)P and PI 4,5-bisphosphate [PI(4,5)P₂], although only PI(4,5)P₂ is believed to be the physiological cellular substrate. Phosphorylation of PI(4,5)P₂ produces the lipid second messenger PI(3,4,5)P₃. More distantly related members of the lipid kinase superfamily are Class IV kinases such as mTOR (discussed above) and DNA-dependent kinase that phosphorylate serine/threonine residues within protein substrates. The most studied and understood of the PI3K lipid kinases are the Class I PI3K enzymes.

Class I PI3 Ks are heterodimers consisting of a p110 catalytic subunit and a regulatory subunit. The family is further divided into Class Ia and Class Ib enzymes on the basis of regulatory partners and the mechanism of regulation. Class Ia enzymes consist of three distinct catalytic subunits (p110α, p110β and p110δ) that dimerise with five distinct regulatory subunits (p85α, p55α, p500α, p85β and p55γ), with all catalytic subunits being able to interact with all regulatory subunits to form a variety of heterodimers. Class Ia PI3Ks are generally activated in response to growth factor-stimulation of receptor tyrosine kinases via interaction of their regulatory subunit SH2 domains with specific phospho-tyrosine residues of activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor systems (GPCRs) and its expression appears to be limited to leukocytes and cardiomyocytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh et al., Nature Genetics, 1999, 21, 99-102) and cervix (Ma et al., Oncogene, 2000, 19, 2739-2744). More recently, activating mutations within the catalytic site of the p110α catalytic subunit have been associated with various other tumours such as those of the colorectal region and of the breast and lung (Samuels et al, Science, 2004, 304, 554). Tumour-related mutations in the p85α regulatory subunit have also been identified in cancers such as those of the ovary and colon (Philp et al., Cancer Research, 2001, 61, 7426-7429). In addition to direct effects, it is believed that activation of Class Ia PI3Ks contributes to tumourigenic events that occur upstream in signalling pathways, for example by way of ligand-dependent or ligand-independent activation of receptor tyrosine kinases, GPCR systems or integrins (Vara et al., Cancer Treatment Reviews, 2004, 30, 193-204). Examples of such upstream signalling pathways include over-expression of the receptor tyrosine kinase erbB2 in a variety of tumours leading to activation of PI3K-mediated pathways (Harari et al., Oncogene, 2000, 19, 6102-6114) and over-expression of the ras oncogene (Kauffmann-Zeh et al., Nature, 1997, 385, 544-548). In addition, Class Ia PI3Ks may contribute indirectly to tumourigenesis caused by various downstream signalling events. For example, loss of the effect of the PTEN tumour-suppressor phosphatase that catalyses conversion of PI(3,4,5)P₃ back to PI(4,5)P₂ is associated with a very broad range of tumours via deregulation of PI3K-mediated production of PI(3,4,5)P₃ (Simpson and Parsons, Exp. Cell Res., 2001, 264, 29-41). Furthermore, augmentation of the effects of other PI3K-mediated signalling events is believed to contribute to a variety of cancers, for example by activation of Akt (Nicholson and Anderson, Cellular Signalling, 2002, 14, 381-395).

In addition to a role in mediating proliferative and survival signalling in tumour cells, there is evidence that Class Ia PI3K enzymes contribute to tumourigenesis in tumour-associated stromal cells. For example, PI3K signalling is known to play an important role in mediating angiogenic events in endothelial cells in response to pro-angiogenic factors such as VEGF (Abid et al., Arterioscler. Thromb. Vasc. Biol., 2004, 24, 294-300). As Class I PI3K enzymes are also involved in motility and migration (Sawyer, Expert Opinion Investig. Drugs, 2004, 13, 1-19), PI3K enzyme inhibitors should provide therapeutic benefit via inhibition of tumour cell invasion and metastasis. In addition, Class I PI3K enzymes play an important role in the regulation of immune cells contributing to pro-tumourigenic effects of inflammatory cells (Coussens and Werb, Nature, 2002, 420, 860-867).

These findings suggest that pharmacological inhibitors of Class I PI3K enzymes will be of therapeutic value for the treatment of various diseases including different forms of the disease of cancer comprising solid tumours such as carcinomas and sarcomas and the leukaemias and lymphoid malignancies. In particular, inhibitors of Class I PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

PI3Kγ, the Class Ib PI3K, is activated by GPCRs, as was finally demonstrated in mice lacking the enzyme. Thus, neutrophils and macrophages derived from PI3Kγ-deficient animals failed to produce PI(3,4,5)P₃ in response to stimulation with various chemotactic substances (such as IL-8, C5a, fMLP and MIP-1a), whereas signalling through protein tyrosine kinase-coupled receptors to Class Ia PI3Ks was intact (Hirsch et al., Science, 2000, 287(5455), 1049-1053; Li et al., Science, 2002, 287(5455), 1046-1049; Sasaki et al., Science 2002, 287(5455), 1040-1046). Furthermore, PI(3,4,5)P₃-mediated phosphorylation of PKB was not initiated by these GPCR ligands in PI3Kγ-null cells. Taken together, the results demonstrated that, at least in resting haematopoietic cells, PI3Kγ is the sole PI3K isoform that is activated by GPCRs in vivo. When murine bone marrow-derived neutrophils and peritoneal macrophages from wild-type and PI3Kγ^{-/-} mice were tested in vitro, a reduced, but not completely abrogated, performance in chemotaxis and adherence assays was observed. However, this translated into a drastic impairment of IL-8 driven neutrophil infiltration into tissues (Hirsch et al., Science, 2000, 287(5455), 1049-1053.). Recent data suggest that PI3Kγ is involved in the path finding process rather than in the generation of mechanical force for motility, as random migration was not impaired in cells that lacked PI3Kγ (Hannigan et al., Proc. Nat. Acad. of Sciences of U.S.A., 2002, 99(6), 3603-8). Data linking PI3Kγ to respiratory disease pathology came with the demonstration that PI3Kγ has a central role in regulating endotoxin-induced lung infiltration and activation of neutrophils leading to acute lung injury (Yum et al., J. Immunology, 2001, 167(11), 6601-8). The fact that although PI3Kγ is highly expressed in leucocytes, its loss seems not to interfere with haematopoiesis, and the fact that PI3Kγ-null mice are viable and fertile further implicates this PI3K isoform as a potential drug target. Work with knockout mice also established that PI3Kγ is an essential amplifier of mast cell activation (Laffargue et al., Immunity, 2002, 16(3), 441-451).

Thus, in addition to tumourigenesis, there is evidence that Class I PI3K enzymes play a role in other diseases (Wymann et al., Trends in Pharmacological Science, 2003, 24, 366-376). Both Class Ia PI3K enzymes and the single Class Ib enzyme have important roles in cells of the immune system (Koyasu, Nature Immunology, 2003, 4, 313-319) and thus they are therapeutic targets for inflammatory and allergic indications. Recent reports demonstrate that mice deficient in PI3Kγ and PI3Kδ are viable, but have attenuated inflammatory and allergic responses (Ali et al., Nature, 2004, 431(7011), 1007-11). Inhibition of PI3K is also useful to treat cardiovascular disease via anti-inflammatory effects or directly by affecting cardiac myocytes (Prasad et al., Trends in Cardiovascular Medicine, 2003, 13, 206-212). Thus, inhibitors of Class I PI3K enzymes are expected to be of value in the prevention and treatment of a wide variety of diseases in addition to cancer.

Several compounds that inhibit PI3Ks and phosphatidylinositol (PI) kinase-related kinase (PI3KKs) have been identified, including wortmannin and the quercetin derivative LY294002. These compounds are reasonably specific inhibitors of PI3Ks and PI3KKs over other kinases but they lack potency and display little selectivity within the PI3K families.

Accordingly, it would be desirable to provide further effective mTOR and/or PI3K inhibitors for use in the treatment of cancer, inflammatory or obstructive airways diseases, immune or cardiovascular diseases.

Molpholino pyrimidine derivatives and PI3K inhibitors are known in the art.

International Patent Application WO 2004/048365 discloses compounds that possess PI3K enzyme inhibitory activity and are useful in the treatment of cancer. These compounds are arylamino- and heteroarylamino-substituted pyrimidines which differ from the compounds of the present invention with respect to their arylamino- and heteroarylamino substituents. These substituents are not equivalent to the —XR¹ substituents of the present invention. Inhibitors of PI3K activity useful in the treatment of cancer are also disclosed in European Patent Application 1 277 738 which mentions 4-morpholino-substituted bicyclic heteroaryl compounds such as quinazoline and pyrido[3,2-d]pyrimidine derivatives and 4-morpholino-substituted tricyclic heteroaryl compounds but not monocyclic pyrimidine derivatives.

A number of compounds such as 4-morpholin-4-yl-6-(phenylsulfonylmethyl)-2-pyridin-4-yl-pyrimidine and 4-{6-[(phenylsulfonyl)methyl]-2-pyridin-2-ylpyrimidin-4-yl}morpholine have been registered in the Chemical Abstracts database but no utility has been indicated and there is no suggestion that these compounds have mTOR and/or PI3K inhibitory activity or useful therapeutic properties.

Surprisingly, we have found that certain morpholino pyrimidine derivatives possess useful therapeutic properties. Without wishing to be bound by theoretical constraints, it is believed that the therapeutic usefulness of the derivatives is derived from their inhibitory activity against mTOR kinase and/or one or more PI3K enzyme (such as the Class Ia enzyme and/or the Class Ib enzyme). Because signalling pathways mediated by the PI3K/mTOR families have a central role in a number of cell processes including proliferation and survival, and because deregulation of these pathways is a causative factor in a wide spectrum of human cancers and other diseases, it is expected that the derivatives will be therapeutically useful. In particular, it is expected that the derivatives will have to anti-proliferative and/or apoptotic properties which means that they will be useful in the treatment of proliferative disease such as cancer. The compounds of the present invention may also be useful in inhibiting the uncontrolled cellular proliferation which arises from various non-malignant diseases such as inflammatory diseases, obstructive airways diseases, immune diseases or cardiovascular diseases.

Generally, the compounds of the present invention possess potent inhibitory activity against mTOR kinase but the compound may also possess potent inhibitory activity against one or more PI3K enzyme (such as the Class Ia enzyme and/or the Class Ib enzyme).

In accordance with an aspect of the present invention, there is provided a compound of formula (I)

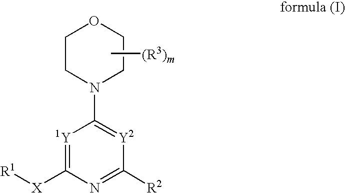

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹²R¹², —NR¹¹COR¹², and —NR¹¹COCONR¹²R¹⁶; each R³, when present, is independently selected from halo, cyano, nitro, —R¹³, —OR¹³, —SR¹³, —SOR¹³, —SO₂R¹³, —COR¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is to optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl,
for use as a medicament in the treatment of proliferative disease.

In accordance with another aspect of the present invention, there is provided a compound of formula (I)

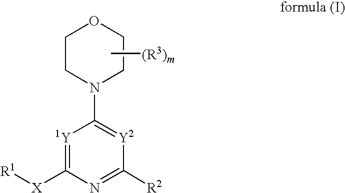

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰; or X—R¹ is —CR⁶R⁷OH;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with another aspect of the present invention, there is provided a compound of formula (I)

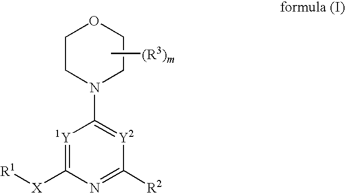

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰; or X—R¹ is —CR⁶R⁷OH;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, 20 C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl for use as a medicament in the treatment of proliferative disease.

In accordance with another aspect of the present invention, there is provided the use of a compound of formula (I)

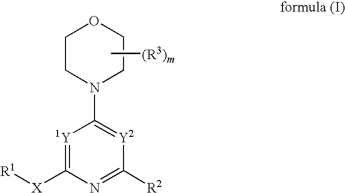

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷, —NR⁴C(O)NR⁵CR⁶R⁷, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is substituted by —NR¹⁷SO₂R's and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl,
in the manufacture of a medicament for use in the treatment of proliferative disease.

In accordance with another aspect of the present invention, there is provided the use of a compound of formula (I)

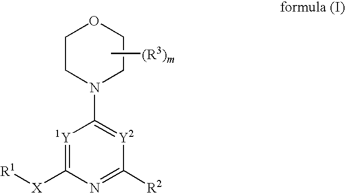

or a pharmaceutically acceptable salt thereof, wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰; or X—R¹ is —CR⁶R⁷OH;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, amino C₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with another aspect of the present invention, there is provided the use of a compound of formula (I)

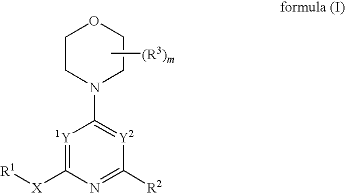

or a pharmaceutically acceptable salt; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;

### or X—R¹ is —CR⁶R⁷OH

R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³—SOR¹³, —SO₂R¹³, —COR¹³, —CO₂R¹³, —CONR¹³R¹⁴, —NR¹³R¹⁴, —NR¹³COR¹⁴, —R¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl in the manufacture of a medicament for use in the treatment of proliferative disease.

In accordance with a further aspect of the present invention, there is also provided a compound of formula (I)

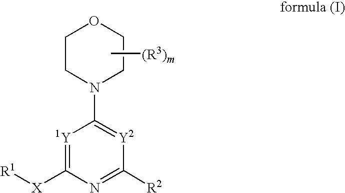

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —(O)₂NR⁴— and —NR⁴S(O)₂—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹²COR¹², and —NR¹¹COCONR¹²R¹⁶;
each R³, when present, is independently selected from halo, cyano, nitro, —R¹³, —OR¹³, —R¹³, —SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In accordance with a further aspect of the present invention, there is also provided a compound of formula (I)

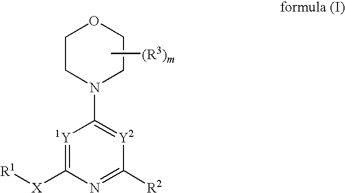

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;

### or X—R¹ is —CR⁶R⁷OH;

R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³—OR¹³, —R¹³, ⁻SOR¹³, —SO₂R¹³, —COR¹³—CO₂R¹³—CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In accordance with a further aspect of the present invention, there is also provided a compound of formula (I)

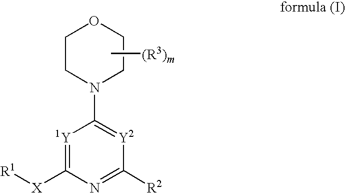

or a pharmaceutically acceptable salt thereof; wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, NR⁴C(O)—, —NR⁴C(O)NR⁵—, —S(O)₂NR⁴— and —NR⁴S(O)₂—;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;

### or X—R¹ is —CR⁶R⁷OH;

R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —R¹³, —SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention. Solvates and mixtures thereof also form an aspect of the present invention. For example, a suitable solvate of a compound of formula (I) is, for example, a hydrate such as a hemi-hydrate, a mono-hydrate, a di-hydrate or a tri-hydrate or an alternative quantity thereof.

The present invention relates to the compounds of formula (I) as herein defined as well as to salts thereof. Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of compounds of formula (I) as herein defined which are sufficiently basic to form such salts. Such acid addition salts include but are not limited to furmarate, methanesulfonate, hydrochloride, hydrobromide, citrate and maleate salts and salts formed with phosphoric and sulfuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl, 1,3-dioxolen-2-onylmethyl esters for example 5-methyl-1,3-dioxolen-2-onylmethyl, and C₁₋₆alkoxycarbonyloxyethyl esters for example 1-methoxycarbonyloxyethyl; and may be formed at any carboxy group in the compounds of this invention.

Suitable pharmaceutically acceptable esters for hydroxy include inorganic esters such as phosphate esters (including phosphoramidic cyclic esters) and (X-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl, benzoyl, phenylacetyl, substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁₋₄alkyl, or phenyl. Suitable substituents on a phenyl group in such esters include, for example, 4-C₁₋₄piperazino-C₁₋₄alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

In this specification the generic term “C_{p-q}alkyl” includes both straight-chain and branched-chain alkyl groups. However references to individual alkyl groups such as “propyl” are specific for the straight chain version only (i.e. 17-propyl and isopropyl) and references to individual branched-chain alkyl groups such as “tert-butyl” are specific for the branched chain version only.

The prefix C_{p-q} in C_{p-q}alkyl and other terms (where p and q are integers) indicates the range of carbon atoms that are present in the group, for example C₁₋₄alkyl includes C₁alkyl (methyl), C₂alkyl (ethyl), C₃alkyl (propyl as n-propyl and isopropyl) and C₄alkyl (n-butyl, sec-butyl, isobutyl and tert-butyl).

The term C_{p-q}alkoxy comprises —O—C_{p-q}alkyl groups.

The term C_{p-q}alkanoyl comprises —C(O)alkyl groups.

The term halo includes fluoro, chloro, bromo and iodo.

“Carbocyclyl” is a saturated, unsaturated or partially saturated monocyclic, bicyclic or tricyclic ring system containing from 3 to 14 ring atoms, wherein a ring CH₂ group may be replaced with a C═O group. “Carbocyclyl” includes “aryl”, “C_{p-q}cycloalkyl” and “C_{p-q}cycloalkenyl”.

“aryl” is an aromatic monocyclic, bicyclic or tricyclic carbcyclyl ring system.

“C_{p-q}cycloalkenyl” is an unsaturated or partially saturated monocyclic, bicyclic or tricyclic carbocyclyl ring system containing at least 1 C═C bond and wherein a ring CH₂ group may be replaced with a C═O group.

“C_{p-q}cycloalkyl” is a saturated monocyclic, bicyclic or tricyclic carbocyclyl ring system and wherein a ring CH₂ group may be replaced with a C═O group.

“Heterocyclyl” is a saturated, unsaturated or partially saturated monocyclic, bicyclic or tricyclic ring system containing from 3 to 14 ring atoms of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group. “Heterocyclyl” includes “heteroaryl”, “cycloheteroalkyl” and “cycloheteroalkenyl”.

“Heteroaryl” is an aromatic monocyclic, bicyclic or tricyclic heterocyclyl, particularly having 5 to 10 ring atoms, of which 1, 2, 3 or 4 ring atoms are chosen from nitrogen, sulfur or oxygen where a ring nitrogen or sulfur may be oxidised.

“Cycloheteroalkenyl” is an unsaturated or partially saturated monocyclic, bicyclic or tricyclic heterocyclyl ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

“Cycloheteroalkyl” is a saturated monocyclic, bicyclic or tricyclic heterocyclic ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

This specification may make use of composite terms to describe groups comprising more than one functionality. Unless otherwise described herein, such terms are to be interpreted as is understood in the art. For example carbocyclylC_{p-q}alkyl comprises C_{p-q}alkyl substituted by carbocyclyl, heterocyclylC_{p-q}alkyl comprises C_{p-q}alkyl substituted by heterocyclyl, and bis(C_{p-q}alkyl)amino comprises amino substituted by 2 C_{p-q}alkyl groups which may be the same or different.

HaloC_{p-q}alkyl is a C_{p-q}alkyl group that is substituted by 1 or more halo substituents and particularly 1, 2 or 3 halo substituents. Similarly, other generic terms containing halo such as haloC_{p-q}alkoxy may contain 1 or more halo substituents and particularly 1, 2 or 3 halo substituents.

HydroxyC_{p-q}alkyl is a C_{p-q}alkyl group that is substituted by 1 or more hydroxyl substituents and particularly by 1, 2 or 3 hydroxy substituents. Similarly other generic terms containing hydroxy such as hydroxyC_{p-q}alkoxy may contain 1 or more and particularly 1, 2 or 3 hydroxy substituents.

C_{p-q}alkoxyC_{p-q}alkyl is a C_{p-q}alkyl group that is substituted by 1 or more C_{p-q}alkoxy substituents and particularly 1, 2 or 3 C_{p-q}alkoxy substituents. Similarly other generic terms containing C_{p-q}alkoxy such as C_{p-q}alkoxyC_{p-q}alkoxy may contain 1 or more C_{p-q}alkoxy substituents and particularly 1, 2 or 3 C_{p-q}alkoxy substituents.

Where optional substituents are chosen from “1 or 2”, from “1, 2, or 3” or from “1, 2, 3 or 4” groups or substituents it is to be understood that this definition includes all substituents being chosen from one of the specified groups i.e. all substitutents being the same or the substituents being chosen from two or more of the specified groups i.e. the substitutents not being the same.

Compounds of the present invention have been named with the aid of computer software (ACD/Name version 8.0).

“Proliferative disease(s)” includes malignant disease(s) such as cancer as well as non-malignant disease(s) such as inflammatory diseases, obstracutive airways diseases, immune diseases or cardiovascular diseases.

Suitable values for any R group or any part or substitutent for such groups include:
for C₁₋₄alkyl: methyl, ethyl, propyl, butyl, 2-methylpropyl and tert-butyl;
for C₁₋₆alkyl: C₁₋₄alkyl, pentyl, 2,2-dimethylpropyl, 3-methylbutyl and hexyl;
for C₃₋₆cycloalkyl: cyclopropyl, cyclobutyl, cyclopentyl and cyclohexyl;
for C₃₋₆cycloalkylC₁₋₄alkyl: cyclopropylmethyl, cyclopropylethyl, cyclobutylmethyl, cyclopentylmethyl and cyclohexylmethyl;
for aryl: phenyl and naphthyl;
for arylC₁₋₄alkyl: benzyl, phenethyl, naphthylmethyl and naphthylethyl;
for carbocylyl: aryl, cyclohexenyl and C₃₋₆cycloalkyl;
for halo: fluoro, chloro, bromo and iodo;
for C₁₋₄alkoxy: methoxy, ethoxy, propoxy and isopropoxy;
for C₁₋₆alkoxy: C₁₋₄alkoxy, pentyloxy, 1-ethylpropoxy and hexyloxy;
for C₆alkanoyl: acetyl, propanoyl and 2-methylpropanoyl;
for heteroaryl: pyridyl, imidazolyl, quinolinyl, cinnolyl, pyrimidinyl, thienyl, pyrrolyl, pyrazolyl, thiazolyl, thiazolyl, triazolyl, oxazolyl, isoxazolyl, furanyl, pyridazinyl, pyrazinyl, indolyl, benzofuranyl, dibenzofuranyl and benzothienyl;
for heteroarylC₁₋₄alkyl: pyrrolylmethyl, pyrrolylethyl, imidazolylmethyl, imidazolylethyl, pyrazolylmethyl, pyrazolylethyl, furanylmethyl, furanylethyl, thienylmethyl, theinylethyl, pyridylmethyl, pyridylethyl, pyrazinylmethyl, pyrazinylethyl, pyrimidinylmethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl;
for heterocyclyl: heteroaryl, pyrrolidinyl, isoquinolinyl, quinoxalinyl, benzothiazolyl, benzoxazolyl, piperidinyl, piperazinyl, azetidinyl, morpholinyl, tetrahydroisoquinolinyl, tetrahydroquinolinyl, indolinyl, dihydro-2H-pyranyl and tetrahydrofuranyl.

It should be noted that examples given for terms used in the description are not limiting.

Particular values of m, X, ¹Y and Y², R¹, R² and R³ are as follows. Such values may be used where appropriate, in connect with any aspect of the invention, or part thereof, and with any of the definitions, claims or embodiments defined herein.

m

In one aspect of the invention m is 0, 1, 2 or 3.

In another aspect m is 0, 1 or 2.

In a further aspect m is 0 or 1.

In yet another aspect m is 0 so that R³ is absent.

In yet another aspect m is 1 and R³ is methyl.

¹Y and Y²

In one aspect of the invention ¹Y is N and Y² is CR⁸.

In another aspect ¹Y is N and Y² is CH.

In yet another aspect ¹Y is CR⁸ and Y² is N.

In a further aspect ¹Y is CH or CF and Y² is N.

In yet a further aspect ¹Y is CH and Y² is N.

X

In one aspect of the invention X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —C(O)NR⁴—, —S(O)₂NR⁴— and —NR⁴S(O)₂—.

In another aspect X is a linker group selected from —NR⁴CR⁶R⁷—, —OCR⁶R⁷, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷, —C(O)NR⁴— and —NR⁴C(O)—.

In a further aspect X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴—, and —NR⁴C(O)—.

In a further aspect X is a linker group selected from —NR⁴CR⁶R⁷—, —OCR⁶R⁷—, —SCR⁶R⁷—, —S(O)CR⁶R⁷— and —S(O)₂CR⁶R⁷—.

In yet another aspect X is a linker group selected from —SCR⁶R⁷—, —S(O)CR⁶R⁷— and —S(O)₂CR⁶R⁷—.

In another aspect X is a linker group selected from —NR⁴—CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)—, —S(O)₂C(CH₃)₂—, —C(O)NR⁴— and —NR⁴C(O)—.

In another aspect X is a linker group selected from —NR⁴—CH₂—, —OCH₂—, —SCH₂—, —S(O)CH₂—, —S(O)₂CH₂—, —C(O)NR⁴—, and —NR⁴C(O)—.

In another aspect X is a linker group selected from —NR⁴—CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)— and —S(O)₂C(CH₃)₂—.

In another aspect X is a linker group selected from —NR⁴—CH₂—, —OCH₂—, —SCH₂—, —S(O)CH₂— and —S(O)₂CH₂—.

In a further aspect X is a linker group selected from —NHCH₂—, —N(CH₃)CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)—, —S(O)₂C(CH₃)₂—, —C(O)NH—, —C(O)N(CH₃)—, —NHC(O)— and —N(CH₃)C(O)—.

In a further aspect X is a linker group selected from —NHCH₂—, —N(CH₃)CH₂—, —OCH₂—, —SCH₂—, —S(O)CH₂—, —S(O)₂CH₂—, —C(O)NH—, —C(O)N(CH₃)—, —NHC(O)— and —N(CH₃)C(O)—.

In yet a further aspect X is a linker group selected from —NHCH₂—, —N(CH₃)CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)— and —S(O)₂C(CH₃)₂—.

In yet a further aspect X is a linker group selected from —NHCH₂—, —N(CH₃)CH₂—, —OCH₂—, —SCH₂— and —S(O)₂CH₂—.

In another aspect X is —SCH₂— or —S(O)₂CH₂—.

In another aspect X is —SCH₂—, —SCH(CH₃)— or —SC(CH₃)₂—.

In another aspect X is —S(O)CH₂—, —S(O)CH(CH₃)— or —S(O)C(CH₃)₂—.

In another aspect X is —S(O)₂CH₂—, —S(O)₂CH(CH₃)— or —S(O)₂C(CH₃)₂—.

In another aspect X is —S(O)₂CH₂—.

In another aspect X is —S(O)₂C(CH₃)₂—.

### R¹

In one aspect of the invention R¹ is a group selected from C₁₋₄alkyl, C₃₋₁₀cycloalkyl, aryl, C₃₋₁₀cycloalkylC₁₋₄alkyl, arylC₁₋₄alkyl, cycloheteroalkyl, heteroaryl, cycloheteroalkylC₁₋₄alkyl, heteroalylC₁₋₄alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰.

In another aspect, R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is to optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰.

In a further aspect, R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃.

In yet another aspect R¹ is a group selected from methyl, isopropyl, cyclopropyl, cyclohexyl, —CH₂CH₂OH, —, —CH₂CH₂NC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrrolidin-3-yl, thiazol-2-yl, 4-methylthiazol-2-yl, and 3-methyl-1,3,4-thiadiazol-2-yl.

In yet another aspect R¹ is methyl.

X—R¹

In one embodiment X—R¹ is —C(CH₃)₂OH or —CH₂OH.

In one embodiment X—R¹ is —CH₂OH.

R²

In one aspect of the invention R² is selected from carbocyclyl or heterocyclyl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from carbocyclyl or heterocyclyl which group is substituted by —NHSO₂R¹⁸ and optionally substituted by one or more substituent group independently selected from halo, cyano, nitro, —R¹¹, —OR¹¹, —COR¹¹, CONR¹¹R¹², —NR¹¹R¹² and —NR¹¹COR¹².

In one aspect of the invention R² is selected from 5 or 6 membered carbocyclyl or heterocyclyl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from 5 or 6 membered carbocyclyl or heterocyclyl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from a 6 membered aryl and 5 or 6 membered heteroaryl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In one aspect of the invention R² is selected from a 6 membered aryl and 5 or 6 membered heteroaryl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, pyrrolyl, imidazolyl, pyrazolyl, furanyl, thienyl, pyridinyl, pyrimidinyl, pyridazinyl, thiazolyl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²—.

In another aspect R² is selected from phenyl, pyrrolyl, imidazolyl, pyrazolyl, furanyl, thienyl, pyridinyl, pyrimidinyl, pyridazinyl, thiazolyl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, pyrrolyl, imidazolyl, pyrazolyl, furanyl, thienyl, pyridinyl, pyrimidinyl, pyridazinyl, thiazolyl which group is substituted by —NR¹⁷SO₂R¹⁸ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is selected from phenyl, pyrrolyl, imidazolyl, pyrazolyl, furanyl, thienyl, pyridinyl, pyrimidinyl, pyridazinyl, thiazolyl which group is substituted by —NHSO₂R¹⁸ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridyl substituted by —NR¹⁷SO₂R¹⁸ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridyl substituted by —NHSO₂R¹⁸ and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl substituted by —NHSO₂R's and optionally substituted by one or more substituent group independently selected from fluoro, methyl, methoxy, hydroxymethyl, cyanomethyl, —CONH₂, —CONHCH₃ and —CON(CH₃)₂.

In another aspect R² is phenyl or pyridyl optionally substituted by —NR¹⁷SO₂R¹⁸.

In another aspect R² is phenyl or pyridyl optionally substituted by —NHSO₂R¹⁸.

In another aspect R² is

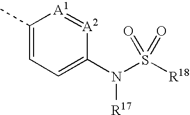

wherein A¹ and A² are selected from CH or N provided that at least one of A¹ or A² is CH.

In another aspect R² is

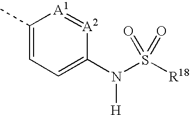

wherein A¹ and A² are selected from CH or N provided that at least one of A¹ or A² is CH.

R⁴

In one aspect of the invention R⁴ is hydrogen or methyl.

In another aspect R⁴ is hydrogen.

R⁴ and R¹

In another aspect of the invention, when X is —NR⁴CR⁶R⁷—, —NR⁴C(O)CR⁶R⁷—, —NR⁴C(O)NR⁵CR⁶R⁷—, —NR⁴S(O)₂CR⁶R⁷—, —NR⁴C(O)—, —NR⁴C(O)NR⁵— or —NR⁴S(O)₂—, R¹ and R⁴ together with the atom or atoms to which they are attached form a 5- to 10-membered heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention, when X is —NR⁴CR⁶R⁷—, —NR⁴C(O)CR⁶R⁷—, —NR⁴C(O)NR⁵CR⁶R⁷—, —NR⁴S(O)₂CR⁶R⁷—, —NR⁴C(O)—, —NR⁴C(O)NR⁵— or —NR⁴S(O)₂—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

R⁵

In one aspect of the invention R⁵ is hydrogen or methyl.

In another aspect R⁵ is hydrogen.

In another aspect R⁵ is methyl.

R⁶

In one aspect of the invention R⁶ is hydrogen or methyl.

In another aspect R⁶ is hydrogen.

In another aspect R⁶ is methyl.

R⁷

In one aspect of the invention R⁷ is hydrogen or methyl.

In another aspect R⁷ is hydrogen.

In another aspect R⁷ is methyl.

R⁸

In one aspect of the invention R⁸ is hydrogen or halo.

In another aspect R⁸ is hydrogen or fluoro.

In a further aspect R⁸ is hydrogen.

R⁹

In one aspect of the invention R⁹ is hydrogen or C₁₋₄alkyl optionally substituted by 1, 2 or 3 substituent groups selected from halo, cyano, nitro, hydroxy, C₁₋₄alkoxy, amino, C₁₋₄alkylamino and bis(C₁₋₄alkyl)amino.

In another aspect R⁹ is hydrogen or C₁₋₄alkyl optionally substituted by 1, 2 or 3 halo substituents.

In a further aspect R⁹ is hydrogen, methyl or trifluoromethyl.

R¹⁰

In one aspect of the invention R¹⁰ is hydrogen.

R¹¹

In one aspect of the invention R¹¹ is hydrogen or a group selected from C₁₋₄alkyl, aryl and cycloheteroalkyl which group is optionally substituted by 1, 2 or 3 groups selected from halo, hydroxy and cyano.

In another aspect R¹¹ is hydrogen, methyl optionally substituted with hydroxy or cyano, phenyl or pyrrolidinyl.

In another aspect R¹¹ is hydrogen or methyl.

R¹²

In one aspect of the invention R¹² is hydrogen or methyl.

R¹⁷

In one aspect of the invention R¹⁷ is hydrogen or a group selected from C₁₋₄alkyl, aryl and cycloheteroalkyl which group is optionally substituted by 1, 2 or 3 groups selected from halo, hydroxy and cyano.

In another aspect R¹⁷ is hydrogen, methyl optionally substituted with hydroxy or cyano, phenyl or pyrrolidinyl.

In another aspect R¹⁷ is hydrogen or methyl.

In another aspect R¹⁷ is hydrogen.

R¹⁸

In one aspect of the invention R¹⁸ is hydrogen or a group selected from C₁₋₆alkyl, C₃₋₆cycloalkyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁸ is hydrogen or a group selected from C₁₋₆alkyl, C₃₋₆cycloakyl, phenyl, napthyl, pyrrolyl, imidazolyl, isoxazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl, phenylC₁₋₆alkyl, naphthylC₁₋₆alkyl, pyrrolylC₁₋₆alkyl, imidazolylC₁₋₆alkyl, isoxazolylC₁₋₆alkyl, pyrazolylC₁₋₆alkyl, furanylC₁₋₆alkyl, thienylC₁₋₆alkyl, pyridinylC₁₋₆alkyl, pyrimidinylC₁₋₆alkyl, pyridazinylC₁₋₆alkyl, azaindolylC₁₋₆alkyl, indolylC₁₋₆alkyl, quinolinylC₁₋₆alkyl, benzimidazolylC₁₋₆alkyl, benzofuranylC₁₋₆alkyl, dibenzofuranylC₁₋₆alkyl, benzothienylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In one aspect of the invention R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-fluorophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, 6-methoxypryridin-3-yl, 5-fluoropyridin-2-yl, pyrimidin-2-yl, and 1H-pyrazol-3-yl.

In one aspect of the invention R¹⁸ is hydrogen or a group selected from methyl, ethyl, propyl, butyl, cyclopropyl, and 4-fluorophenyl.

In one aspect of the invention there is provided a subset of compounds of formula (I), or a pharmaceutically acceptable salt thereof;

m is 0, 1 or 2;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —S(O)₂NR⁴— and —NR⁴S(O)₂—;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;

### or X—R¹ is —C(CH₃)₂OH or —CH₂OH;

R² is selected from aryl and heteroaryl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
each R³, when present, is methyl;
R⁴ and R⁵ are independently hydrogen or C₁₋₆alkyl;
or, when X is —NR⁴CR⁶R⁷—, —NR⁴C(O)NR⁵CR⁶R⁷—, —NR⁴C(O)— or —NR⁴S(O)₂—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁷ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
and
R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (I), or a pharmaceutically acceptable salt thereof;

m is 0, 1 or 2;
¹Y and Y² are independently N or CR⁸ provided that one of ¹Y and Y² is N and the other is CR⁸;
X is a linker group selected from —NR⁴—CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)—, —S(O)₂C(CH₃)₂—, —C(O)NR⁴— and —NR⁴C(O)—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰; or X—R¹ is —C(CH₃)₂OH or —CH₂OH;
R² is selected from 5 or 6 membered aryl and heteroaryl which group is substituted by —NHSO₂R¹⁸ and optionally substituted by one or more substituent group independently selected from halo, cyano, nitro, —R¹², —OR¹¹, —COR¹¹, —CONR¹¹R¹², —NR¹¹R¹² and —NR¹¹COR¹²;
each R³, when present, is methyl;
R⁴ is hydrogen or C₁₋₆alkyl;
or, when X is —NR⁴—CH₂— or —NR⁴C(O)—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (I), or a pharmaceutically acceptable salt thereof;

m is 0 or 1;

### ¹Y is CH and Y² is N;

X is a linker group selected from —S(O)₂CH₂—, —S(O)₂CH(CH₃)— and —S(O)₂C(CH₃)₂—;
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;

### or —XR¹ is C(CH₃)₂OH or —CH₂OH;

R² is selected from phenyl, pyrrolyl, imidazolyl, pyrazolyl, furanyl, thienyl, pyridinyl, pyrimidinyl, pyridazinyl and thiazolyl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³, when present, is methyl;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;

m is 1;
X is a linker group selected from —S(O)₂CH₂—, —S(O)₂CH(CH₃)— and —S(O)₂C(CH₃)₂—;

### ¹Y is CH and Y² is N.

R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
R² is phenyl or pyridyl substituted by —NHSO₂R¹⁸ and optionally substituted by one or more substituent group independently selected from fluoro, methyl, methoxy, hydroxymethyl, cyanomethyl, —CONH₂, —CONHCH₃ and —CON(CH₃)₂;
R³ is methyl; and
R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (I), or a pharmaceutically acceptable salt thereof;

m is 1;
X is a linker group selected from —S(O)₂CH₂—, —S(O)₂CH(CH₃)— and —S(O)₂C(CH₃)₂—;

### ¹Y is CH and Y² is N.

R¹ is a group selected from methyl, isopropyl, cyclopropyl, cyclohexyl, —CH₂CH₂OH, —CH₂CH₂NC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrrolidin-3-yl, thiazol-2-yl, 4-methylthiazol-2-yl, and 3-methyl-1,3,4-thiadiazol-2-yl;

### R² is

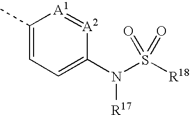

wherein
A¹ and A² are selected from CH or N provided that at least one of A¹ or A² is CH;
R¹⁷ is hydrogen;
R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-fluorophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, 6-methoxypryridin-3-yl, 5-fluoropyridin-2-yl, pyrimidin-2-yl, and 1H-pyrazol-3-yl;

and, R³ is methyl.

In one aspect of the invention there is provided a subset of compounds of formula (Ia) or (Ib)

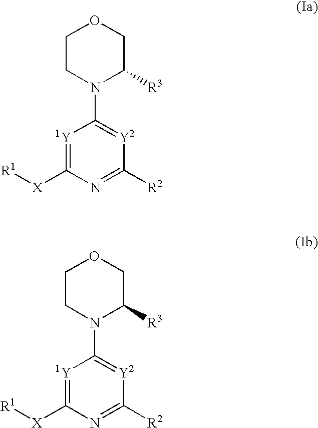

or a pharmaceutically acceptable salt thereof;
¹Y and Y² are independently N or CR⁸ provided that one of ¹Y and Y² is N and the other is CR⁸;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —S(O)₂NR⁴— and —NR⁴S(O)₂—;
R¹ is a group selected from C₁₋₆alkyl, carbocyclyl, carbocyclylC₁₋₆alkyl, heterocyclyl and to heterocyclylC₁₋₆alkyl, which group is optionally substituted by one or more substitutent group selected from halo, cyano, nitro, R⁹, —OR⁹, —COR⁹, —CONR⁹R¹⁰, —NR⁹R¹⁰ and —NR⁹COR¹⁰;

### or X—R¹ is —C(CH₃)₂OH or —CH₂OH;

R² is selected from aryl and heteroaryl which group is substituted by —NR¹⁷SO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁴ and R⁵ are independently hydrogen or C₁₋₆alkyl
or when X is —NR⁴CR⁶R⁷—, —NR⁴C(O)NR⁵CR⁶R⁷—, —NR⁴C(O)— or —NR⁴S(O)₂—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹, R¹² and R¹⁷ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
and
R¹³ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another aspect of the invention there is provided a subset of compounds of formula (Ia) or (Ib)

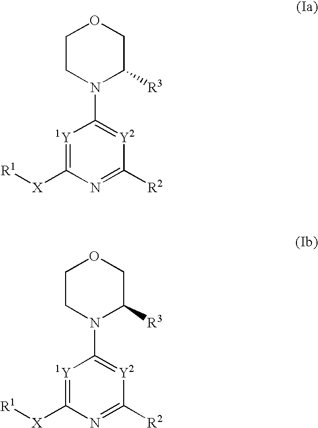

or a pharmaceutically acceptable salt thereof;
¹Y and Y² are independently N or CR⁸ provided that one of ¹Y and Y² is N and the other is CR⁸;
X is a linker group selected from —NR⁴—CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)—, —S(O)₂C(CH₃)₂—, —C(O)NR⁴— and —NR⁴C(O)—;
R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰; or X—R¹ is —C(CH₃)₂OH or —CH₂OH;
R² is selected from 5 or 6 membered aryl and heteroaryl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R⁴ is hydrogen or C₁₋₆alkyl;
or, when X is —NR⁴—CH₂— or —NR⁴C(O)—, R¹ and R⁴ together with the atom or atoms to which they are attached form a 5- or 6-membered heterocyclic ring wherein 1 ring carbon atom is optionally replaced with N or O and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In another particular class of compound of formula (Ia) or (Ib),

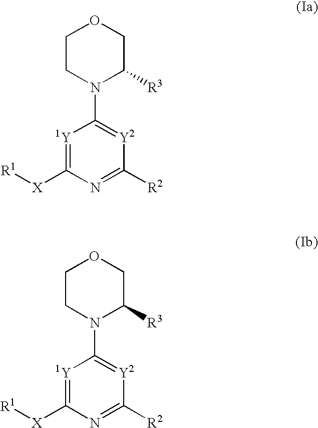

or a pharmaceutically acceptable salt thereof;

### ¹Y is CH and Y² is N;

X is a linker group selected from —S(O)₂CH₂—, —S(O)₂CH(CH₃)— and —S(O)₂C(CH₃)₂—;
R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, to halo, cyano, methyl, methoxy, trifluoromethyl, trifluoromethoxy, —NHCOCH₃, —CONH₂ and —CONHCH₃;

### or —XR¹ is —C(CH₃)₂OH or —CH₂OH;

R² is selected from phenyl, pyrrolyl, imidazolyl, pyrazolyl, furanyl, thienyl, pyridinyl, pyrimidinyl, pyridazinyl and thiazolyl which group is substituted by —NHSO₂R¹⁸ and optionally substituted by one or more substituent group independently selected from halo, cyano, nitro, —R¹¹, —OR¹¹, —COR¹¹, —CONR¹¹R¹², —NR¹¹R¹² and —NR¹¹COR¹²;
R³ is methyl;
R¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino; and
R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆allyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

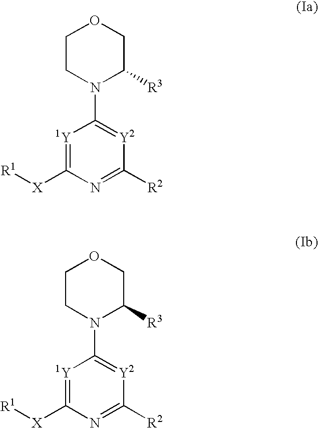

or a pharmaceutically acceptable salt thereof;
X is a linker group selected from —S(O)₂CH₂—, —S(O)₂CH(CH₃)— and —S(O)₂C(CH₃)₂—;

### ¹Y is CH and Y² is N.

R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent group selected from amino, halo, cyano, methyl, methoxy, trifluoromethyl, trifluoromethoxy, —NHCOCH₃, —CONH₂ and —CONHCH₃;
R² is phenyl or pyridyl substituted by —NHSO₂R¹⁸ and optionally substituted by one or more substituent group independently selected from fluoro, methyl, methoxy, hydroxymethyl, cyanomethyl, —CONH₂, —CONHCH₃ and —CON(CH₃)₂;
R³ is methyl; and
R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆allyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

In a further particular class of compound of formula (Ia) or (Ib)

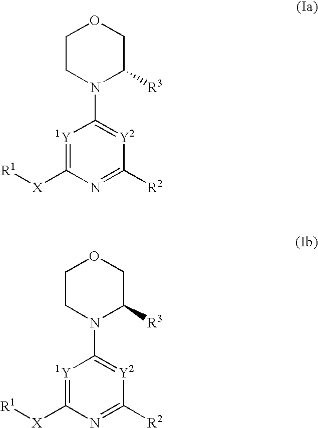

or a pharmaceutically acceptable salt thereof;
m is 1;
X is a linker group selected from —S(O)₂CH₂—, —S(O)₂CH(CH₃)— and —S(O)₂C(CH₃)₂—;

### ¹Y is CH and Y² is N.

R¹ is a group selected from methyl, isopropyl, cyclopropyl, cyclohexyl, —CH₂CH₂OH, —CH₂CH₂NC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrrolidin-3-yl, thiazol-2-yl, 4-methylthiazol-2-yl, and 3-methyl-1,3,4-thiadiazol-2-yl;

### R² is

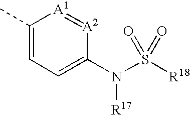 [0215] wherein
A¹ and A² are selected from CH or N provided that at least one of A¹ or A² is CH;
   R¹⁷ is hydrogen; and
   R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-fluorophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, 6-methoxypryridin-3-yl, 5-fluoropyridin-2-yl, pyrimidin-2-yl, and 1H-pyrazol-3-yl;
      and, R³ is methyl.

Another aspect of the invention provides a compound, or a combination of compounds, selected from any one of the Examples or a pharmaceutically acceptable salt thereof.

The invention also provides processes for the preparation of a compound of formula (I) or a pharmaceutically acceptable salt thereof.

A compound of formula (I), wherein X═—S(O)₂CR⁶R⁷—, may be prepared by oxidising a compound of the formula (I), wherein X═SCR⁶R⁷—, for example by using Oxone® at room temperature in a mixed solvent system of water and ethanol

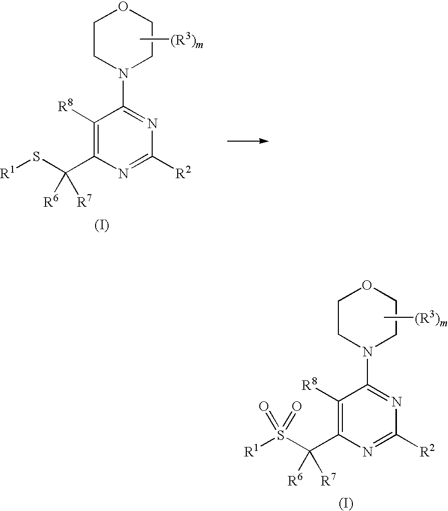

A compound of formula (I), wherein R¹X═R¹OCR⁶R⁷—, may be prepared by the reaction of a compound of formula (I), wherein R¹X═HOCR⁶R⁷—, with a compound of formula (II), wherein L¹ is a leaving group (such as halo, tosyl, mesyl etc.) optionally in the presence of a suitable base such as triethylamine and a solvent such as tetrahydrofuran or N,N-dimethylformamide.

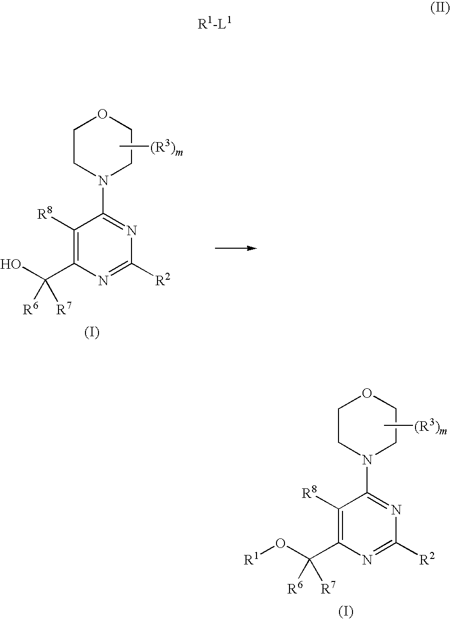

A compound of formula (I), wherein R¹X═R¹R⁴NCR⁶R⁷—, may be prepared by the reaction of a compound of formula (I), wherein R¹X═HR⁴NCR⁶R⁷—, with a compound of formula (II), wherein L¹ is a leaving group (such as halo, tosyl, mesyl etc.) optionally in the presence of a suitable base such as triethylamine and a solvent such as tetrahydrofuran or N,N-dimethylformamide; or by the reaction of a compound of formula (I), wherein R¹X═HR⁴NCR⁶R⁷—, with a compound of formula (III) in the presence of a suitable reducing agent such as NaCNBH₃.

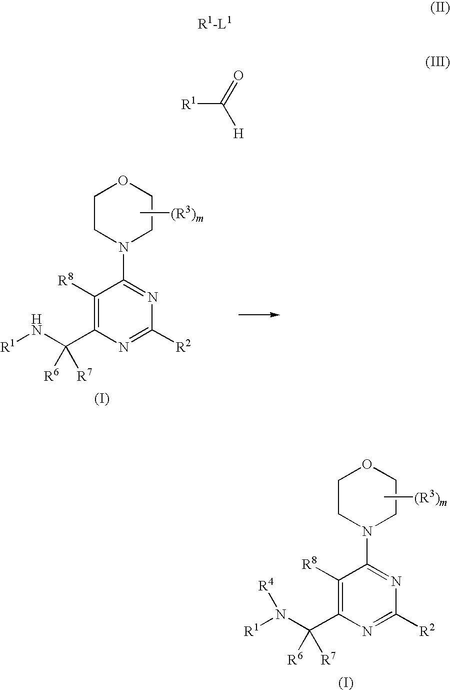

A compound of formula (I), wherein X¹=—S(O)₂CR⁶R⁷—, —SCR⁶R⁷—, —OCR⁶R⁷—, —R⁴NCR⁶R⁷—, —S(O)CR⁶R⁷—, may be prepared by the reaction of a compound of formula (IV), wherein L¹ is a leaving group (such as halo, tosyl, mesyl etc.), with a compound of formula (V) optionally in the presence of a suitable base such as triethylamine and a solvent such as tetrahydrofuran or N,N-dimethylformamide.

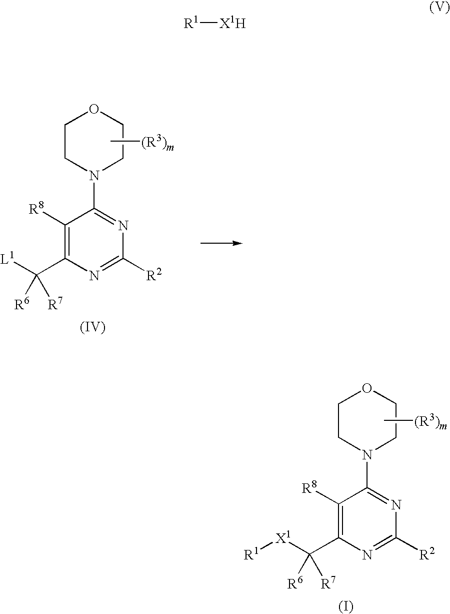

A compound of formula (I), wherein X═—SCR⁶R⁷—, may be prepared by the reaction of a compound of formula (IV), wherein L¹ is a leaving group (such as halo, tosyl, mesyl etc.), with thiourea in a suitable solvent such as ethanol to generate a compound of formula (VI) which is then subsequently reacted with a compound of formula (II) in the presence of a suitable base such as sodium hydroxide and a solvent such as N,N-dimethylformamide.

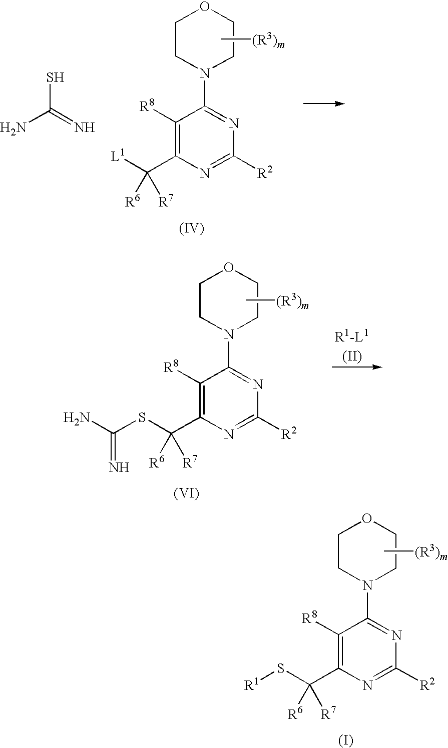

A compound of formula (I), wherein X═—R⁴NC(O)—, may be prepared by the reaction of a compound of formula (VII) with an amine of formula R¹R⁴NH following the suitable activation of the carboxylic acid by methods known in the literature such as the use of a coupling agent such as HATU or the conversion to an acyl chloride.

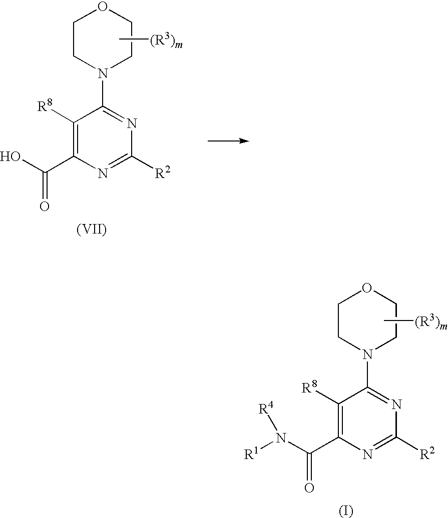

A compound of formula (I), wherein X═—S(O)₂CR⁶R⁷—, may be prepared by the sequential reaction of a compound of formula (I), wherein X═—S(O)₂CH₂—, with a compound of formula (VIII) followed by reaction with a compound of formula (IX), wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide.

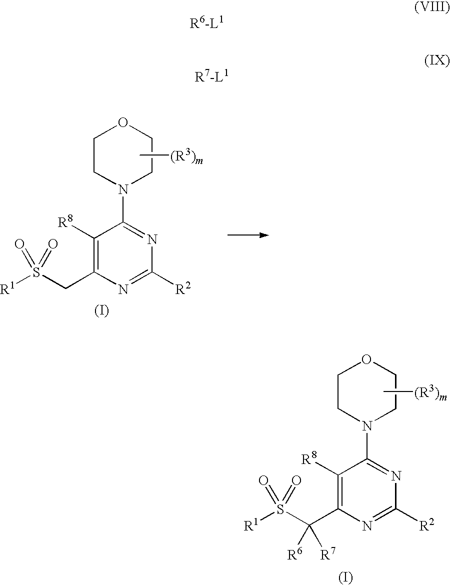

A compound of formula (I), wherein R¹X═HOCR⁶R⁷—, may be prepared by the reaction of a compound of formula (X), with suitable organometallic reagents of formula (XI) and formula (XII) such as the grignard reagent in a suitable solvent. Where R⁶ and R⁷ are different then it may be possible to use techniques known in the literature such the conversion of a compound of formula (X) to the Weinreb amide and reaction with an organometallic reagent of formula (XI) and then reaction with an organometallic reagent of formula (XII) in a subsequent step.

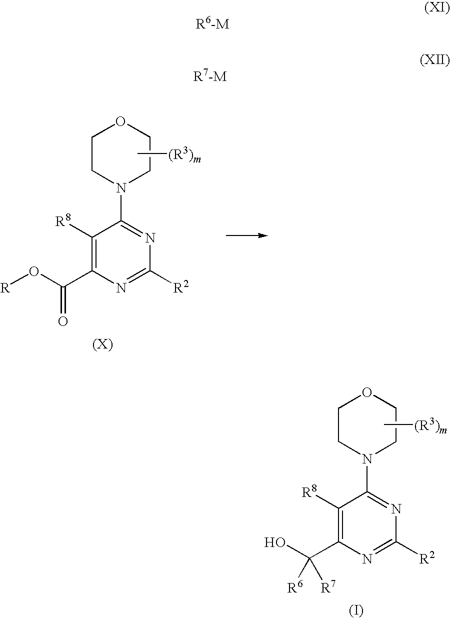

A compound of formula (I) may be prepared from a compound of formula (XIII), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (I) may be prepared from a compound of formula (XIII), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

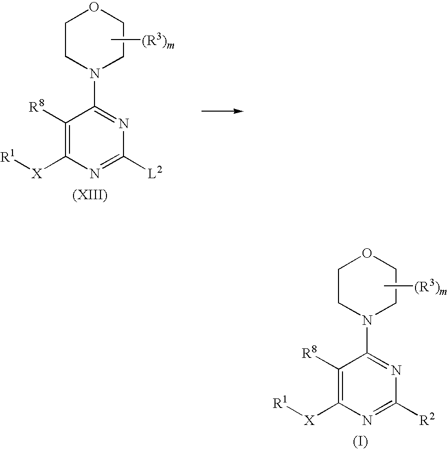

It will be appreciated that a compound of formula (XIII) may be transformed into another compound of formula (XIII) by techniques such as oxidation, alkylation, reductive amination etc., either listed above or otherwise known in the literature.

A compound of formula (XIII), wherein X¹=—S(O)₂CR⁶R⁷—, —SCR⁶R⁷—, —OCR⁶R⁷—, —R⁴NCR⁶R⁷—, —S(O)CR⁶R⁷—, may be prepared by the reaction of a compound of formula (XIV), wherein L¹ is a leaving group (such as halo, tosyl, mesyl etc.), with a compound of formula (V) optionally in the presence of a suitable base such as triethylamine and a solvent such as tetrahydrofuran or N,N-dimethylformamide.

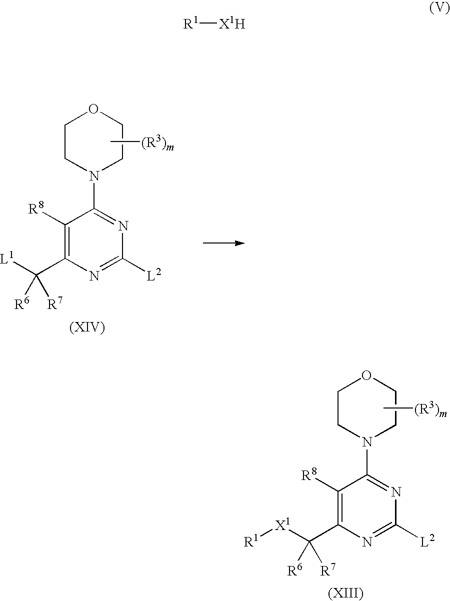

A compound of formula (XIII), wherein X═—SCR⁶R⁷—, may be prepared by the reaction of a compound of formula (XIV), wherein L¹ is a leaving group (such as halo, tosyl, mesyl etc.), with thiourea in a suitable solvent such as ethanol to generate a compound of formula (XV) which is then subsequently reacted with a compound of formula (II) in the presence of a suitable base such as sodium hydroxide and a solvent such as N,N-dimethylformamide.

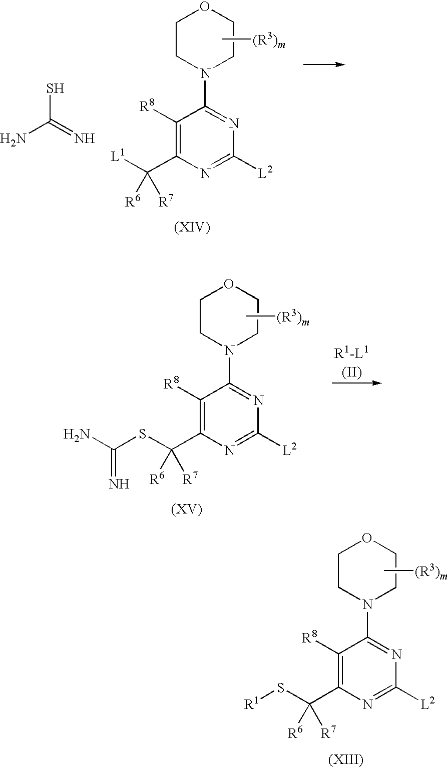

A compound of formula (XIII), wherein X═—R⁴NC(O)—, may be prepared by the reaction of a compound of formula (XVI) with an amine of formula R¹R⁴NH following the suitable activation of the carboxylic acid by methods known in the literature such as the use of a coupling agent such as HATU or the conversion to an acyl chloride.

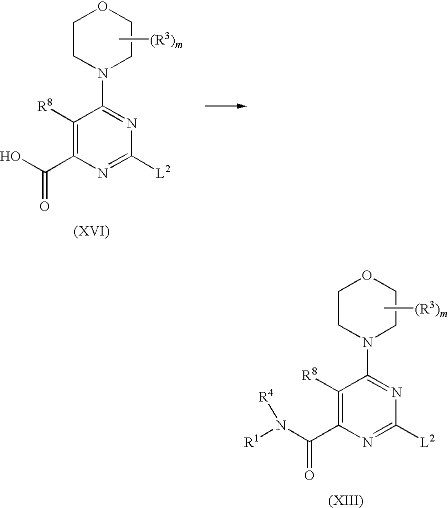

A compound of formula (XIII), wherein X═—S(O)₂CR⁶R⁷—, may be prepared by the sequential reaction of a compound of formula (XIII), wherein X═—S(O)₂CH₂—, with a compound of formula (VIII) followed by reaction with a compound of formula (IX), wherein L¹ is a leaving group (such as halo, tosyl, mesyl etc.), in the presence of a suitable base such as sodium hydride or potassium tert-butoxide in a suitable solvent such as tetrahydrofuran or N,N-dimethylformamide.

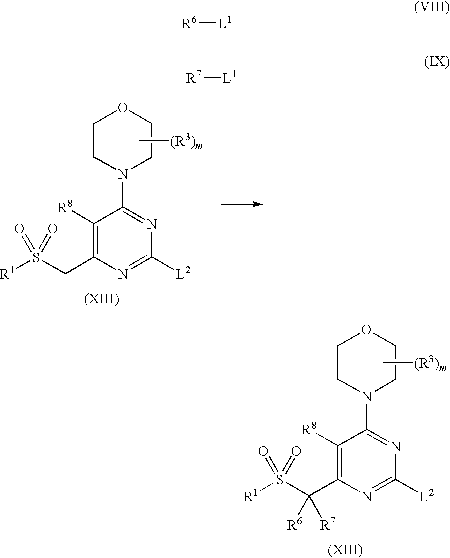

A compound of formula (XIII), wherein R¹X═HOCR⁶R⁷—, may be prepared by the reaction of a compound of formula (XVII), with suitable organometallic reagents of formula (XI) and formula (XII) such as the grignard reagent in a suitable solvent. Where R⁶ and R⁷ are different then it may be possible to use techniques known in the literature such the conversion of a compound of formula (XVII) to the Weinreb amide and reaction with an organometallic reagent of formula (XI) and then reaction with an organometallic reagent of formula (XII) in a subsequent step.

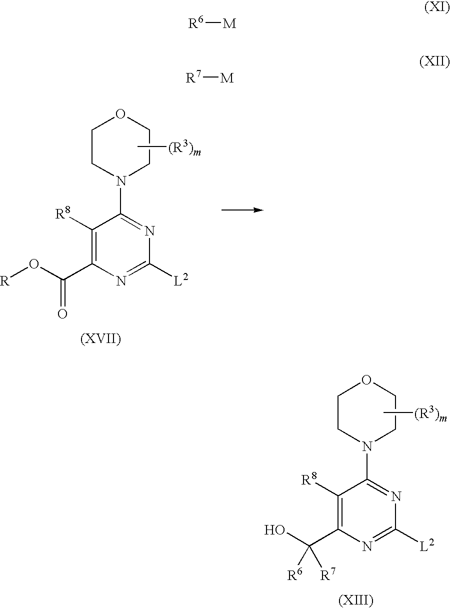

A compound of formula (IV) may be prepared from a compound of formula (XIV), wherein L² is a leaving group (such as halo, tosyl, mesyl, —SMe, —S(O)₂Me etc.) and L¹ is a leaving group (such as halo, tosyl, mesyl etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (IV) may be prepared from a compound of formula (XIV), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

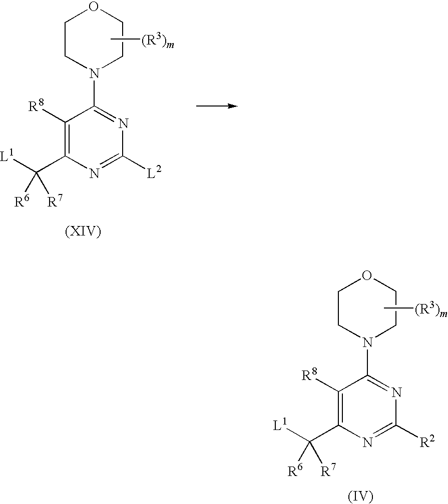

A compound of formula (X) may be prepared from a compound of formula (XVII), wherein L² is a leaving group (such as halo, tosyl, mesyl, —SMe, —S(O)₂Me etc.) and R is a hydrogen or C₁₋₄ alkyl group, with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (X) may be prepared from a compound of formula (XVII), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

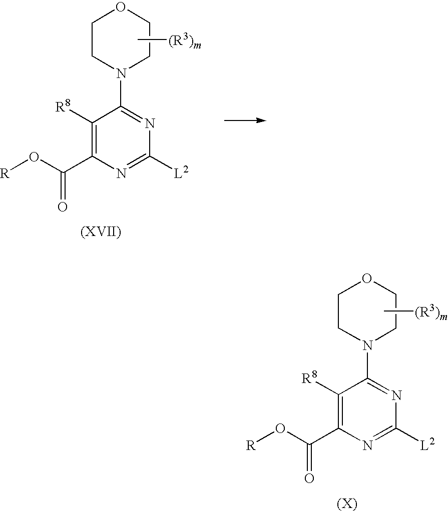

A compound of formula (XVIII) may be prepared from a compound of formula (XIX), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (XVIII) may be prepared from a compound of formula (XIX), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

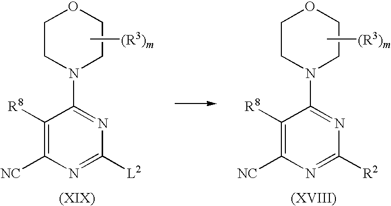

A compound of formula (XX) may be prepared from a compound of formula (XXI), wherein L² is a leaving group (such as halo, tosyl, mesyl, —SMe, —S(O)₂Me etc.), with a suitable organometallic reagent (such as the boronic acid R²B(OH)₂ or the boronic ester R²B(OR)₂ etc.) in the presence of a suitable metal catalyst (such as palladium or copper) in a suitable solvent such as 1,4-dioxane. Alternatively where R² connects to the pyrimidine ring through a nitrogen, oxygen or sulphur atom a compound of formula (XX) may be prepared from a compound of formula (XXI), wherein L² is a leaving group (such as halo, tosyl, mesyl, —SMe, —S(O)₂Me etc.), by reaction with the required amine, alcohol or thiol in the presence of a suitable base such as potassium carbonate in a suitable solvent such as N,N-dimethylformamide.

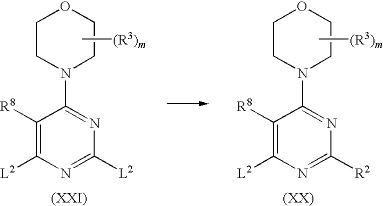

A compound of formula (I), wherein L¹ is a leaving group (such as halo, tosyl, mesyl etc.), may be prepared by the reaction of a compound of formula (XXII) with a compound of formula (XXIII) optionally in the presence of a suitable base such as triethylamine in a suitable solvent such as N,N-dimethylformamide.

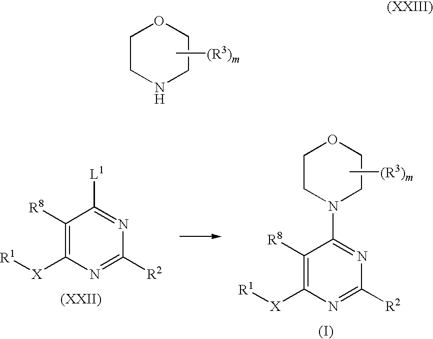

It will be appreciated that a compound of formula (XXII) may be transformed into another compound of formula (XXII) by techniques such as oxidation, alkylation, reductive amination etc., either listed above or otherwise known in the literature.

A compound of formula (IV), wherein L¹ is a leaving group (such as halo, tosyl, mesyl etc.), may be prepared by the reaction of a compound of formula (XXIV) with a compound of formula (XXIII) optionally in the presence of a suitable base such as triethylamine in a suitable solvent such as N,N-dimethylformamide.

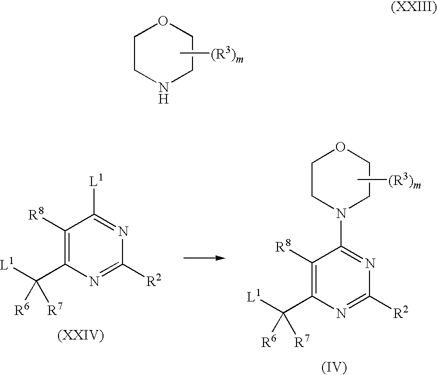

A compound of formula (X), wherein L¹ is a leaving group (such as halo, tosyl, mesyl etc.) and R is a hydrogen or a C₁₋₄ alkyl group, may be prepared by the reaction of a compound of formula (XXV) with a compound of formula (XXIII) optionally in the presence of a suitable base such as triethylamine in a suitable solvent such as N,N-dimethylformamide.

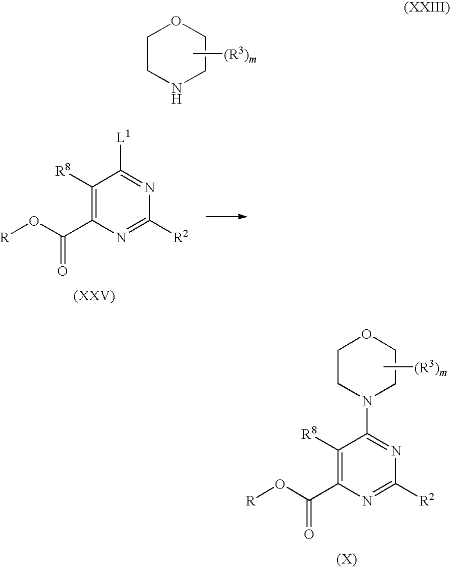

A compound of formula (XVIII), wherein L¹ is a leaving group (such as halo, tosyl, mesyl etc.), may be prepared by the reaction of a compound of formula (XXVI) with a compound of formula (XXIII) optionally in the presence of a suitable base such as triethylamine in a suitable solvent such as N,N-dimethylformamide.

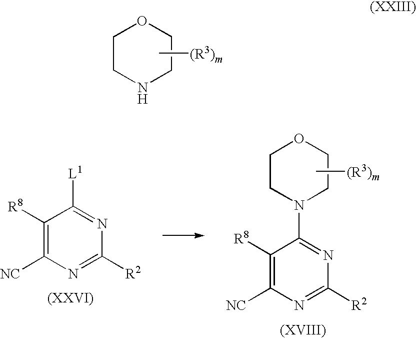

A compound of formula (XX), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXVII) with a compound of formula (XXIII) optionally in the presence of a suitable base such as triethylamine in a suitable solvent such as N,N-dimethylformamide.

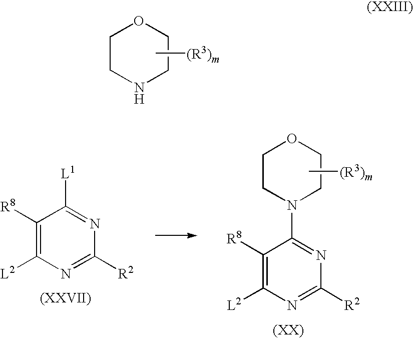

A compound of formula (XIII), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXVIII) with a compound of formula (XXIII) optionally in the presence of a suitable base such as triethylamine in a suitable solvent such as N,N-dimethylformamide.

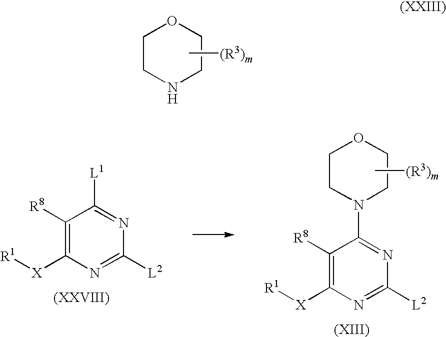

It will be appreciated that a compound of formula (XIII) may be transformed into another compound of formula (XIII) by techniques such as oxidation, alkylation, reductive amination etc., either listed above or otherwise known in the literature.

A compound of formula (XIV), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXIX) with a compound of formula (XXIII) optionally in the presence of a suitable base such as triethylamine in a suitable solvent such as N,N-dimethylformamide.

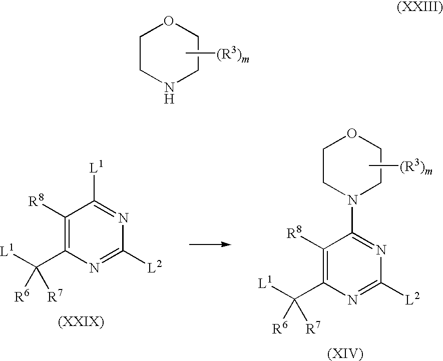

A compound of formula (XVII), wherein L¹ is a leaving group (such as halo, tosyl, mesyl etc.) and L² is a leaving group (such as halo, tosyl, mesyl, —SMe, —S(O)₂Me etc.) and R is a hydrogen or a C₁₋₄ alkyl group, may be prepared by the reaction of a compound of formula (XXX) with a compound of formula (XXIII) optionally in the presence of a suitable base such as triethylamine in a suitable solvent such as N,N-dimethylformamide.

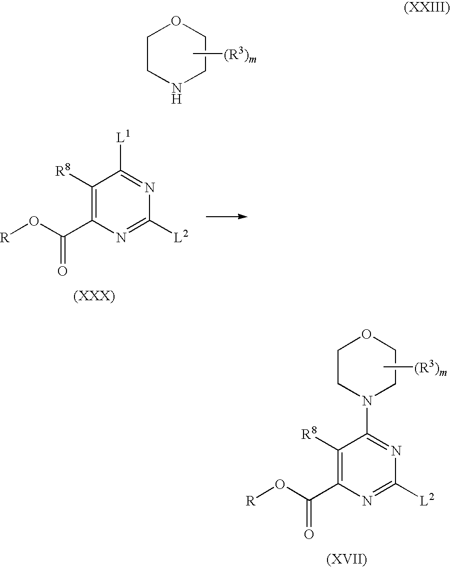

A compound of formula (XIX), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXXI) with a compound of formula (XXIII) optionally in the presence of a suitable base such as triethylamine in a suitable solvent such as N,N-dimethylformamide.

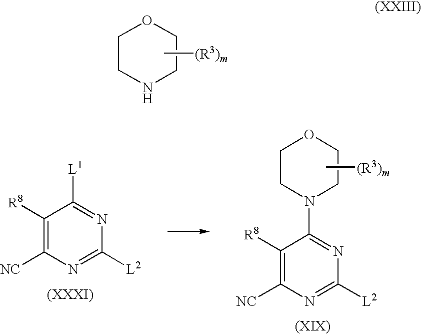

A compound of formula (XXI), wherein L¹ is a leaving group (such as halo, tosyl, mesyl etc.) and L² is a leaving group (such as halo, tosyl, mesyl, —SMe, —S(O)₂Me etc.), may be prepared by the reaction of a compound of formula (XXXII) with a compound of formula (XXIII) optionally in the presence of a suitable base such as triethylamine in a suitable solvent such as N,N-dimethylfomamide.

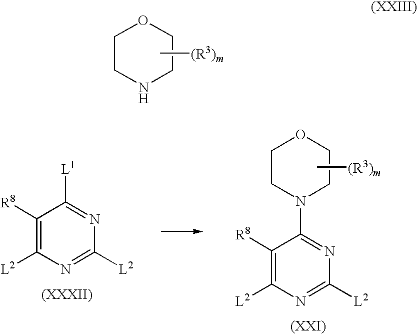

A compound of formula (I), wherein R¹X═H₂NCH₂—, may be prepared from a compound of formula (XVIII) by a reduction such as hydrogenation with hydrogen gas and a suitable catalyst such as Palladium on carbon in a suitable solvent such as ethanol.

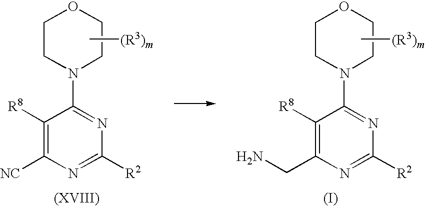

A compound of formula (I), wherein R¹X═H₂NC(O)—, may be prepared from a compound of formula (XVIII) by hydrolysis with, for example, sodium hydroxide in a suitable solvent such as a water ethanol mix.

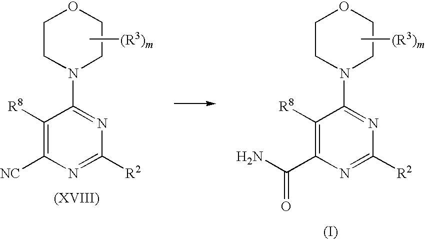

A compound of formula (I), wherein R¹X═H₂NCR⁶R⁷—, may be prepared from a compound of formula (XVIII) by reaction with organometallic reagents (XI) and (XII).

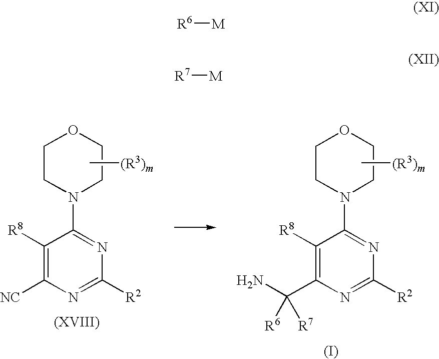

A compound of formula (XIII), wherein R¹X═H₂NCH₂—, may be prepared from a compound of formula (XIX) by a reduction such as hydrogenation with hydrogen gas and a suitable catalyst such as Palladium on carbon in a suitable solvent such as ethanol.

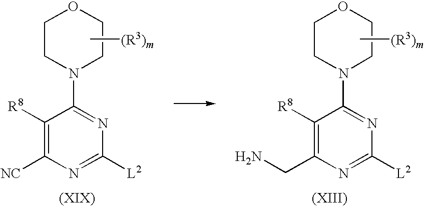

A compound of formula (XIII), wherein R¹X═H₂NC(O)—, may be prepared from a compound of formula (XIX) by hydrolysis with, for example, sodium hydroxide in a suitable solvent such as a water ethanol mix.

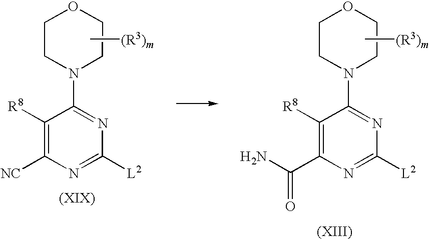

A compound of formula (XIII), wherein R¹X═H₂NCR⁶R⁷—, may be prepared from to a compound of formula (XIX) by reaction with organometallic reagents (XI) and (XII).

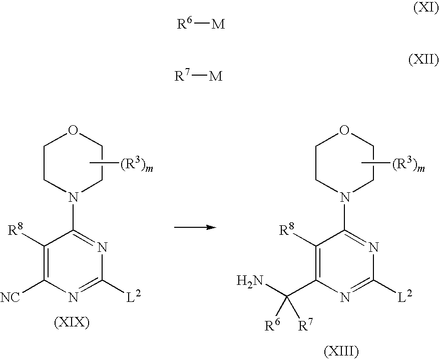

It will be appreciated that the R² group may be introduced at any stage initially as a carbocyclic or heterocyclic amine (optionally with the nitrogen protected, such protecting groups include but are not limited to nitro, tert-butoxy carbamate etc.) which can be transformed at a subsequent stage in the synthesis (after appropriate deprotection) to a sulphonamide by the reaction with a sulphonyl chloride (or other suitably activated species) in the presence of a suitable base, or other methods of forming a sulphonamide known in the literature.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. For example compounds of formula (I) my be converted into further compounds of formula (I) by standard aromatic substitution reactions or by conventional functional group modifications. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an allyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Many of the intermediates defined herein are novel and these are provided as a further feature of the invention.

### Biological Assays

The following assays can be used to measure the effects of the compounds of the present invention as mTOR kinase inhibitors, as PI3 kinase inhibitors, as inhibitors in vitro of the activation of PI3 kinase signalling pathways and as inhibitors in vitro of the proliferation of MDA-MB-468 human breast adenocarcinoma cells.

(a) In Vitro mTOR Kinase Assay

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulfate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one). A 30 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH7.4 buffer (50 mM), EGTA (0.1 mM), bovine serum albumin (0.5 mg/mL), DTT (1.25 mM) and manganese chloride (10 mM)] was agitated at room temperature for 90 minutes.

Control wells that produced a maximum signal corresponding to maximum enzyme activity were created by using 5% DMSO instead of test compound. Control wells that produced a minimum signal corresponding to fully inhibited enzyme were created by adding EDTA (83 mM) instead of test compound. These assay solutions were incubated for 2 hours at room temperature.

Each reaction was stopped by the addition of 10 μl of a mixture of EDTA (50 mM), bovine serum albumin (BSA; 0.5 mg/mL) and Tris-HCl pH7.4 buffer (50 mM) containing

p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left for about 20 hours at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

### (b) In Vitro PI3K Enzyme Assay

The assay used AlphaScreen technology (Gray et al., Analytical Biochemistry, 2003, 313: 234-245) to determine the ability of test compounds to inhibit phosphorylation by recombinant Type I PI3K enzymes of the lipid P1(4,5)P2.

DNA fragments encoding human PI3K catalytic and regulatory subunits were isolated from cDNA libraries using standard molecular biology and PCR cloning techniques. The selected DNA fragments were used to generate baculovirus expression vectors. In particular, full length DNA of each of the p110α, p110β and p110δ Type Ia human PI3K p110 isoforms (EMBL Accession Nos. HSU79143, S67334, Y10055 for p110α, p110β and p110δ respectively) were sub-cloned into a pDEST10 vector (Invitrogen Limited, Fountain Drive, Paisley, UK). The vector is a Gateway-adapted version of Fastbac1 containing a 6-His epitope tag. A truncated form of Type Ib human PI3K p110γ isoform corresponding to amino acid residues 144-1102 (EMBL Accession No. X8336A) and the full length human p85α regulatory subunit (EMBL Accession No. HSP13KIN) were also sub-cloned into pFastBac1 vector containing a 6-His epitope tag. The Type Ia p110 constructs were co-expressed with the p85α regulatory subunit. Following expression in the baculovirus system using standard baculovirus expression techniques, expressed proteins were purified using the His epitope tag using standard purification techniques.

DNA corresponding to amino acids 263 to 380 of human general receptor for phosphoinositides (Grp1) PH domain was isolated from a cDNA library using standard molecular biology and PCR cloning techniques. The resultant DNA fragment was sub-cloned into a pGEX 4T1 E. coli expression vector containing a GST epitope tag (Amersham Pharmacia Biotech, Rainham, Essex, UK) as described by Gray et al., Analytical Biochemistry, 2003, 313: 234-245). The GST-tagged Grp1 PH domain was expressed and purified using standard techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one, Brunel Way, Stonehouse, Gloucestershire, UK Catalogue No. 784075). A mixture of each selected recombinant purified PI3K enzyme (15 ng), DiC8-PI(4,5)P2 substrate (40 μM; Cell Signals Inc., Kinnear Road, Columbus, USA, Catalogue No. 901), adenosine triphosphate (ATP; 4 μM) and a buffer solution [comprising Tris-HCl pH7.6 buffer (40 mM, 10 μl), 3-[(3-cholamidopropyl)dimethylammomio]-1-propanesulfonate (CHAPS; 0.04%), dithiothreitol (DTT; 2 mM) and magnesium chloride (10 nM)] was agitated at room temperature for 20 minutes.

Control wells that produced a minimum signal corresponding to maximum enzyme activity were created by using 5% DMSO instead of test compound. Control wells that produced a maximum signal corresponding to fully inhibited enzyme were created by adding wortmannin (6 μM; Calbiochem/Merck Bioscience, Padge Road, Beeston, Nottingham, UK, Catalogue No. 681675) instead of test compound. These assay solutions were also agitated for 20 minutes at room temperature.

Each reaction was stopped by the addition of 10 μl of a mixture of EDTA (100 nm), bovine serum albumin (BSA, 0.045%) and Tris-HCl pH7.6 buffer (40 mM).

Biotinylated-DiC8-PI(3,4,5)P3 (50 nM; Cell Signals Inc., Catalogue No. 107), recombinant purified GST-Gip1 PH protein (2.5 nM) and AlphaScreen Anti-GST donor and acceptor beads (100 ng; Packard Bioscience Limited, Station Road, Pangbourne, Berkshire, UK, Catalogue No. 6760603M) were added and the assay plates were left for about 5 to 20 hours at room temperature in the dark. The resultant signals arising from laser light excitation at 680 nm were read using a Packard AlphaQuest instrument.

PI(3,4,5)P3 is formed in situ as a result of PI3K mediated phosphorylation of PI(4,5)P2. The GST-Grp1 PH domain protein that is associated with AlphaScreen Anti-GST donor beads forms a complex with the biotinylated PI(3,4,5)P3 that is associated with Alphascreen Streptavidn acceptor beads. The enymatically-produced PI(3,4,5)P3 competes with biotinylated PI(3,4,5)P3 for binding to the PH domain protein. Upon laser light excitation at 680 nm, the donor bead: acceptor bead complex produces a signal that can be measured. Accordingly, PI3K enzyme activity to form PI(3,4,5)P3 and subsequent competition with biotinylated PI(3,4,5)P3 results in a reduced signal. In the presence of a PI3K enzyme inhibitor, signal strength is recovered.

PI3K enzyme inhibition for a given test compound was expressed as an IC₅₀ value.

(c) In Vitro phospho-Ser473 Akt Assay

This assay determines the ability of test compounds to inhibit phosphorylation of Serine 473 in Akt as assessed using Acumen Explorer technology (Acumen Bioscience Limited), a plate reader that can be used to rapidly quantitate features of images generated by laser-scanning.

A MDA-MB-468 human breast adenocarcinoma cell line (LGC Promochem, Teddington, Middlesex, UK, Catalogue No. HTB-132) was routinely maintained at 37° C. with 5% CO₂ up to a confluency of 70-90% in DMEM containing 10% heat-inactivated FCS and 1% L-glutamine.

For the assay, the cells were detached from the culture flask using ‘Accutase’ (Innovative Cell Technologies Inc., San Diego, Calif., USA; Catalogue No. AT104) using standard tissue culture methods and resuspended in media to give 1.7×10⁵ cells per mL. Aliquots (90 μl) were seeded into each of the inner 60 wells of a black Packard 96 well plate (PerkinElmer, Boston, Mass., USA; Catalogue No. 6005182) to give a density of ˜15000 cells per well. Aliquots (90 μl) of culture media were placed in the outer wells to prevent edge effects. The cells were incubated overnight at 37° C. with 5% CO₂ to allow them to adhere.

On day 2, the cells were treated with test compounds and incubated for 2 hours at 37° C. with 5% CO₂. Test compounds were prepared as 10 mM stock solutions in DMSO and serially diluted as required with growth media to give a range of concentrations that were 10-fold the required final test concentrations. Aliquots (10 μl) of each compound dilution were placed in a well (in triplicate) to give the final required concentrations. As a minimum response control, each plate contained wells having a final concentration of 100 μM LY294002 (Calbiochem, Beeston, UK, Catalogue No. 440202). As a maximum response control, wells contained 1% DMSO instead of test compound. Following incubation, the contents of the plates were fixed by treatment with a 1.6% aqueous formaldehyde solution (Sigma, Poole, Dorset, UK, Catalogue No. F1635) at room temperature for 1 hour.

All subsequent aspiration and wash steps were carried out using a Tecan 96 well plate washer (aspiration speed 10 mm/sec). The fixing solution was removed and the contents of the plates were washed with phosphate-buffered saline (PBS; 50 μl; Gibco, Catalogue No. 10010015). The contents of the plates were treated for 10 minutes at room temperature with an aliquot (50 μl) of a cell permeabilisation buffer consisting of a mixture of PBS and 0.5% Tween-20. The ‘permeabilisation’ buffer was removed and non-specific binding sites were blocked by treatment for 1 hour at room temperature of an aliquot (50 μL) of a blocking buffer consisting of 5% dried skimmed milk [‘Marvel’®; Premier Beverages, Stafford, GB] in a mixture of PBS and 0.05% Tween-20. The ‘blocking’ buffer was removed and the cells were incubated for 1 hour at room temperature with rabbit anti phospho-Akt (Ser473) antibody solution (50 μl per well; Cell Signalling, Hitchin, Herts, U.K., Catalogue No 9277) that had been diluted 1:500 in ‘blocking’ buffer. Cells were washed three times in a mixture of PBS and 0.05% Tween-20. Subsequently, cells were incubated for 1 hour at room temperature with Alexafluor488 labelled goat anti-rabbit IgG (50 μl per well; Molecular Probes, Invitrogen Limited, Paisley, UK, Catalogue No. A11008) that had been diluted 1:500 in ‘blocking’ buffer. Cells were washed 3 times with a mixture of PBS and 0.05% Tween-20. An aliquot of PBS (50 μl) was added to each well and the plates were sealed with black plate sealers and the fluorescence signal was detected and analysed.

Fluorescence dose response data obtained with each compound were analysed and the degree of inhibition of Serine 473 in Akt was expressed as an IC₅₀ value.

(d) In Vitro MDA-MB-468 Human Breast adenocarcinoma Proliferation Assay

This assay determines the ability of test compounds to inhibit cell proliferation as assessed using Cellomics Arrayscan technology. A MDA-MB-468 human breast adenocarcinoma cell line (LGC Promochem, Catalogue No. HTB-132) was routinely maintained as described in Biological Assay (b) herein.

For the proliferation assay, the cells were detached from the culture flask using Accutase and seeded into the inner 60 wells of a black Packard 96 well plate at a density of 8000 cells per well in 100 μl of complete growth media. The outer wells contained 100 μl of sterile PBS. The cells were incubated overnight at 37° C. with 5% CO₂ to allow them to adhere.

On day 2, the cells were treated with test compounds and incubated for 48 hours at 37° C. with 5% CO₂. Test compounds were prepared as 10 mM stock solutions in DMSO and serially diluted as required with growth media to give a range of test concentrations. Aliquots (50 μl) of each compound dilution were placed in a well and the cells were incubated for 2 days at 37° C. with 5% CO₂. Each plate contained control wells without test compound.

On day 4, BrdU labelling reagent (Sigma, Catalogue No. B9285) at a final dilution of 1:1000 was added and the cells were incubated for 2 hours at 37° C. The medium was removed and the cells in each well were fixed by treatment with 100 μl of a mixture of ethanol and glacial acetic acid (90% ethanol, 5% glacial acetic acid and 5% water) for 30 minutes at room temperature. The cells in each well were washed twice with PBS (100 μl). Aqueous hydrochloric acid (2M, 100 μl) was added to each well. After 20 minutes at room temperature, the cells were washed twice with PBS. Hydrogen peroxide (3%, 50 μl; Sigma, Catalogue No. H1009) was added to each well. After 10 minutes at room temperature, the wells were washed again with PBS.

BrdU incorporation was detected by incubation for 1 hour at room temperature with mouse anti-BrdU antibody (50 μL; Caltag, Burlingame, Calif., US; Catalogue No. MD5200) that was diluted 1:40 in PBS containing 1% BSA and 0.05% Tween-20. Unbound antibody was removed with two washes of PBS. For visualisation of incorporated BrdU, the cells were treated for 1 hour at room temperature with PBS (50 μl) and 0.05% Tween-20 buffer containing a 1:1000 dilution of Alexa fluor 488-labelled goat anti-mouse IgG. For visualisation of the cell nucleus, a 1:1000 dilution of Hoechst stain (Molecular Probes, Catalogue No. H3570) was added. Each plate was washed in turn with PBS. Subsequently, PBS (100 μl) was added to each well and the plates were analysed using a Cellomics array scan to assess total cell number and number of BrdU positive cells.

Fluorescence dose response data obtained with each compound were analysed and the degree of inhibition of MDA-MB-468 cell growth was expressed as an IC₅₀ value.

Although the pharmacological properties of the compounds of formula (I) vary with structural change as expected, in general, it is believed that activity possessed by compounds of formula (I) may be demonstrated at the following concentrations or doses in one or more of the above tests (a) to (d):—
Test (a): —IC₅₀ versus mTOR kinase at less than 10 μM, in particular less than 5 μM for many compounds, for Example 4 the IC₅₀ was measured on three occasions, the values were 3.32, 2.49 and 2.25 μM;
Test (b): —IC₅₀ versus p110γ Type Ib human PI3K at less than 10 μM; and IC₅₀ versus p110α Type Ia human PI3K at less than 10 μM; For Example 4 the IC₅₀ was measured on three occasions, the values were >134, 54, >698 μM, indicating this compound may be a selective mTOR inhibitor;
Test (c): —IC₅₀ versus Serine 473 in Akt at less than 100 μM, in particular less than 35 μM for many compounds, for Example 4 the IC₅₀ was measured on three occasions, the values were 11.29, 24.05 and >31.88 μM;
Test (d): —IC₅₀ at less than 20 μM;

The compounds of the present invention are advantageous in that they possess pharmacological activity. In particular, the compounds of the present invention modulate (in particular, inhibit) mTOR kinase and/or phosphatidylinositol-3-kinase (PI3K) enzymes, such as the Class Ia PI3K enzymes (e.g. PI3Kalpha, PI3 Kbeta and PI3 Kdelta) and the Class Ib PI3K enzyme (PI3 Kgamma). More particularly compounds of the present invention modulate (in particular, inhibit) mTOR kinase. More particularly compounds of the present invention modulate (in particular, inhibit) one or more PI3K enzyme. The inhibitory properties of compounds of formula (I) may be demonstrated using the test procedures set out herein and in the experimental section. Accordingly, the compounds of formula (I) may be used in the treatment (therapeutic or prophylactic) of conditions/diseases in human and non-human animals which are mediated by mTOR kinase and/or one or more PI3K enzyme(s), and in particular by mTOR kinase.

The invention also provides a pharmaceutical composition comprising a compound of formula (I), or a pharmaceutically acceptable salt thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of formula I will naturally vary according to the nature and severity of the disease state, the age and sex of the animal or patient and the route of administration, according to well known principles of medicine.

In using a compound of formula (I) for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Typically, unit dosage forms will contain about 10 mg to 0.5 g of a compound of this invention.

As stated herein, it is known that mTOR kinase and the PI3K enzymes have roles in tumourigenesis as well as numerous other diseases. We have found that the compounds of formula (I) possess potent anti-tumour activity which it is believed is obtained by way of inhibition of mTOR kinase and/or one or more of the PI3K enzymes.

Accordingly, the compounds of the present invention are of value as anti-tumour agents. Particularly, the compounds of the present invention are of value as anti-proliferative, apoptotic and/or anti-invasive agents in the containment and/or treatment of solid and/or liquid tumour disease. Particularly, the compounds of the present invention are expected to be useful in the prevention or treatment of those tumours which are sensitive to inhibition of mTOR and/or one or more of the PI3K enzymes such as the Class Ia PI3K enzymes and the Class Ib PI3K enzyme. Further, the compounds of the present invention are expected to be useful in the prevention or treatment of those tumours which are mediated alone or in part by mTOR and/or one or more of the PI3K enzymes such as the Class Ia PI3K enzymes and the Class Ib PI3K enzyme. The compounds may thus be used to produce an mTOR enzyme inhibitory effect in a warm-blooded animal in need of such treatment. Certain compounds may be used to produce an PI3K enzyme inhibitory effect in a warm-blooded animal in need of such treatment.

As stated herein, inhibitors of mTOR kinase and/or one or more PI3K enzymes should be of therapeutic value for the treatment of proliferative disease such as cancer and in particular solid tumours such as carcinoma and sarcomas and the leukaemias and lymphoid malignancies and in particular for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias [including acute lymphoctic leukaemia (ALL) and chronic myelogenous leukaemia (CML)], multiple myeloma and lymphomas.

According to a further aspect of the invention there is provided a compound of formula (I), or a pharmaceutically acceptable salt thereof, as defined herein for use as a medicament in a warm-blooded animal such as man.

According to a further aspect of the invention, there is provided a compound of formula (I), or a pharmaceutically acceptable salt thereof, as defined herein for use in the production of an anti-proliferative effect in a warm-blooded animal such as man.

According to a further aspect of the invention, there is provided a compound of formula (I), or a pharmaceutically acceptable salt thereof, as defined herein for use in the production of an apoptotic effect in a warn-blooded animal such as man.

According to a further feature of the invention there is provided a compound of formula (I), or a pharmaceutically acceptable salt thereof, as defined herein for use in a warm-blooded animal such as man as an anti-invasive agent in the containment and/or treatment of proliferative disease such as cancer.

According to a further aspect of the invention, there is provided the use of a compound of formula (I), or a pharmaceutically acceptable salt thereof, as defined herein for the production of an anti-proliferative effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an anti-proliferative effect in a warm-blooded animal such as man.

According to a further aspect of the invention, there is provided the use of a compound of formula (I), or a pharmaceutically acceptable salt thereof, as defined herein for the production of an apoptotic effect in a warm-blooded animal such as man. According to a further feature of this aspect of the invention there is provided the use of a compound of formula (I), or a pharmaceutically acceptable salt thereof, as defined herein in the manufacture of a medicament for use in the production of an apoptotic effect in a warm-blooded animal such as man.

According to a further feature of the invention there is provided the use of a compound of formula (I), or a pharmaceutically acceptable salt thereof, as defined herein in the manufacture of a medicament for use in a warm-blooded animal such as man as an anti-invasive agent in the containment and/or treatment of proliferative disease such as cancer.

According to a further feature of this aspect of the invention there is provided a method for producing an anti-proliferative effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-invasive effect by the containment and/or treatment of solid tumour disease in a warm-blooded animal, such as man, in need of such treatment which comprises administering to said animal an effective amount of a compound of formula (I), or a pharmaceutically acceptable salt thereof, as defined herein.

According to a further aspect of the invention there is provided the use of a compound of formula (I), or a pharmaceutically acceptable salt thereof, as defined herein in the manufacture of a medicament for use in the prevention or treatment of proliferative disease such as cancer in a warm-blooded animal such as man.

According to a further feature of this aspect of the invention there is provided a method for the prevention or treatment of proliferative disease such as cancer in a warm-blooded animal, such as man, in need of such treatment which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells.

According to a further feature of this aspect of the invention there is provided the use of a compound of formula (I), or a pharmaceutically acceptable salt thereof, as defined herein in the manufacture of a medicament for use in the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells.

According to a further feature of this aspect of the invention there is provided a method for the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in providing a mTOR kinase inhibitory effect and/or a PI3K enzyme inhibitory effect (such as a Class Ia PI3K enzyme or Class Ib PI3K enzyme inhibitory effect).

According to a further feature of this aspect of the invention there is provided the use of a compound of formula (I), or a pharmaceutically acceptable salt thereof, as defined herein in the manufacture of a medicament for use in providing a mTOR kinase inhibitory effect and/or a PI3K enzyme inhibitory effect (such as a Class Ia PI3K enzyme or Class Ib PI3K enzyme inhibitory effect).

According to a further aspect of the invention there is also provided a method for providing a mTOR kinase inhibitory effect and/or a PI3K enzyme inhibitory effect (such as a Class Ia PI3K enzyme or Class Ib PI3K enzyme inhibitory effect) which comprises administering an effective amount of a compound of formula I, or a pharmaceutically acceptable salt thereof, as defined herein.

According to a further feature of the invention there is provided a compound of formula I, or a pharmaceutically acceptable salt thereof, as defined herein for use in the treatment of cancer, inflammatory diseases, obstructive airways diseases, immune diseases or cardiovascular diseases.

According to a further feature of the invention there is provided a compound of formula I, or a pharmaceutically acceptable salt thereof, as defined herein for use in the treatment of solid tumours such as carcinoma and sarcomas and the leukaemias and lymphoid malignancies.

According to a further feature of the invention there is provided a compound of formula I, or a pharmaceutically acceptable salt thereof, as defined herein for use in the treatment of cancer of the breast, colorectum, lung (including small cell lung cancer, non-small cell lung cancer and bronchioalveolar cancer) and prostate.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided the use of a compound of formula (I), or a pharmaceutically acceptable salt thereof, as defined herein in the manufacture of a medicament for use in the treatment of cancer, inflammatory diseases, obstructive airways diseases, immune diseases or cardiovascular diseases.

According to a further feature of the invention there is provided the use of a compound of formula (I), or a pharmaceutically acceptable salt thereof, as defined herein in the manufacture of a medicament for use in the treatment of solid tumours such as carcinoma and sarcomas and the leukaemias and lymphoid malignancies.

According to a further feature of the invention there is provided the use of a compound of formula (I), or a pharmaceutically acceptable salt thereof, as defined herein in the manufacture of a medicament for use in the treatment of cancer of the breast, colorectum, lung (including small cell lung cancer, non-small cell lung cancer and bronchioalveolar cancer) and prostate.

According to a further feature of the invention there is provided the use of a compound of formula (I), or a pharmaceutically acceptable salt thereof, as defined herein in the manufacture of a medicament for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided a method for treating cancer, inflammatory diseases, obstructive airways diseases, immune diseases or cardiovascular diseases in a warm blooded animal such as man that is in need of such treatment which comprises administering an effective amount of a compound of formula (I), or a pharmaceutically acceptable salt thereof, as defined herein.

According to a further feature of the invention there is provided a method for treating solid tumours such as carcinoma and sarcomas and the leukaemias and lymphoid malignancies in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the breast, colorectum, lung (including small cell lung cancer, non-small cell lung cancer and bronchioalveolar cancer) and prostate in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas in a warm blooded animal such as man that is in need of such treatment which comprises administering an effective amount of a compound of formula (I), or a pharmaceutically acceptable salt thereof, as defined herein.

As stated herein, the in vivo effects of a compound of formula (I) may be exerted in part by one or more metabolites that are formed within the human or animal body after administration of a compound of formula (I).

The invention further relates to combination therapies wherein a compound of formula (I), or a pharmaceutically acceptable salt thereof, or a pharmaceutical composition or formulation comprising a compound of formula (I) is administered concurrently or sequentially or as a combined preparation with another treatment of use in the control of oncology disease.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment of cancer.

Suitable agents to be used in combination include: —

(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including ex-vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

Unless stated otherwise, starting materials were commercially available. All solvents and commercial reagents were of laboratory grade and were used as received.

In the examples ¹H NMR spectra were recorded on a Bruker DPX 300 (300 MHz), Bruker DRX 400 (400 MHz) instrument or a Bruker DRX 500 (500 MHz) instrument. The central peaks of chloroform-d (δ_{H} 7.27 ppm), dimethylsulfoxide-d₆ (δ_{H} 2.50 ppm) or acetone-d₆ (δ_{H} 2.05 ppm) were used as internal references. The following abbreviations have been used: s, singlet; d, doublet;

t, triplet; q, quartet; m, multiplet; br, broad.

Column chromatography was carried out using silica gel (0.04-0.063 mm, Merck). In general, a Kromasil KR-100-5-C18 reversed-phase column (250×20 mm, Akzo Nobel) was used for preparative HPLC with mixtures of acetonitrile and water [containing 0.1% trifluoroacetic acid (TFA)] used as the eluent at a flow rate of 10 mL/min. The following methods were used for liquid chromatography (LC)/mass spectral (MS) analysis: —

HPLC: Agilent 1100 or Waters Alliance HT (2790 & 2795)

Mass Spectrometer Waters ZQ ESCi

### HPLC Column

The standard HPLC column used is the Phemonenex Gemini C18 5 μm, 50×2 mm.

### Acidic HPLC Methods

| | | |
|---|---|---|
| The mobile phases used are: | Mobile phase A: | Water |
| | Mobile Phase B: | Acetonitrile |
| | Mobile Phase C: | 1% Formic Acid in |
| | | 50:50 Water:MeCN |
| | | (v/v) |

Each method is followed by a rapid equilibration using a 5 mL flow rate for 0.45 min.

### Four Generic HPLC Methods are Available:

5 Minute Monitor Acidic method

| | Mobile | Mobile | Mobile | | Flow |
|---|---|---|---|---|---|
| Time/ | Phase | Phase | Phase | | Rate/ |
| min | A: | B: | C: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

### Early Acidic Method for Early Eluting Compounds

| | Mobile | Mobile | Mobile | | Flow |
|---|---|---|---|---|---|
| Time/ | Phase | Phase | Phase | | Rate/ |
| min | A: | B: | C: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

### Mid Acidic Method for Middle Eluting Compounds

| | Mobile | Mobile | Mobile | | Flow |
|---|---|---|---|---|---|
| Time/ | Phase | Phase | Phase | | Rate/ |
| min | A: | B: | C: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

### Late Acidic Method for Late Eluting Compounds

| | Mobile | Mobile | Mobile | | Flow |
|---|---|---|---|---|---|
| Time/ | Phase | Phase | Phase | | Rate/ |
| min | A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

### Basic HPLC Methods

In some instances the standard acidic methods may be unsuitable for either the compound ionisation or the chromatography separation required. In such cases four comparable Basic HPLC methods are available.

| | | |
|---|---|---|
| The mobile phases used are: | Mobile phase A: | Water |
| | Mobile Phase B: | Acetonitrile |
| | Mobile Phase D: | 0.1% 880 Ammonia in |
| | | acetonitrile |

Each method is followed by a rapid equilibration using a 5 mL flow rate for 0.45 min.

Minute Monitor Basic method

| | Mobile | Mobile | Mobile | | Flow |
|---|---|---|---|---|---|
| Time/ | Phase | Phase | Phase | | Rate/ |
| min | A: | B: | D: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

### Early Basic Method for Early Eluting Compounds

| | Mobile | Mobile | Mobile | | Flow |
|---|---|---|---|---|---|
| Time/ | Phase | Phase | Phase | | Rate/ |
| min | A: | B: | D: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

### Mid Basic Method for Middle Eluting Compounds

| | Mobile | Mobile | Mobile | | Flow |
|---|---|---|---|---|---|
| Time/ | Phase | Phase | Phase | | Rate/ |
| min | A: | B: | D: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

### Late Basic Method for Late Eluting Compounds

| | Mobile | Mobile | Mobile | | Flow |
|---|---|---|---|---|---|
| Time/ | Phase | Phase | Phase | | Rate/ |
| min | A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

The following method was used for liquid chromatography (LC)/mass spectral (MS) analysis:—

Instrument: Agilent 1100; Column: Waters ‘Symmetry’ 2.1×30 mm; Mass Spectral analysis using chemical ionisation (APCI); Flow rate: 0.7 mL/min; Absorption Wavelength: 254 nm; Solvent A: water+0.1% TFA; Solvent B: acetonitrile+0.1% TFA; Solvent Gradient: 15-95% Solvent B for 2.7 minutes followed by 95% Solvent B for 0.3 minutes.

The following methods were used for LC analysis:—

Method A: —Instrument: Agilent 1100; Column: Kromasil C18 reversed-phase silica, 100×3 mm, 5 μm particle size; Solvent A: 0.1% TFA/water, Solvent B: 0.08% TFA/acetonitrile; Flow Rate: 1 mL/min; Solvent Gradient: 10-100% Solvent B for 20 minutes followed by 100% Solvent B for 1 minute; Absorption Wavelengths: 220, 254 and 280 nm. In general, the retention time of the product was noted.

Method B: —Instrument: Agilent 1100; Column: Waters ‘Xterra’ C8 reversed-phase silica, 100×3 mm, 5 μm particle size; Solvent A: 0.01514 ammonia in water, Solvent B: acetonitrile; Flow Rate: 1 ml/min, Solvent Gradient: 10-100% Solvent B for 20 to minutes followed by 100% Solvent B for 1 minute; Absorption Wavelength: 220, 254 and 280 min. In general, the retention time of the product was noted.

The following abbreviations are used herein or within the following illustrative examples:—

### HPLC High Performance Liquid Chromatography

HBTU O-(benzotriazol-1-yl)-N,N,N′,N′-tetramethyluronium hexafluorophosphate;
HATU O-(7-azabenzotriazol-1-yl)-N,N,N′,N′-tetramethyluronium hexafluorophosphate;
HOBT 1-hydroxybenzotriazole;
HOAT 1-hydroxy-7-azabenzotriazole;
NMP N-methylpyrrolidin-2-one;
DMSO dimethylsulfoxide;

### DMF N,N-dimethylformamide;

### DMA N,N-dimethylacetamide;

THF tetrahydrofuran;
DME 1,2-dimethoxyethane;
DCCI dicyclohexylcarbodiimide;
MeOH methanol;
MeCN acetonitrile;
DCM dichloromethane;

### DIPEA N,N-diisopropylethylamine;

DBU 1,8-diazabicyclo[5.4.0]undec-7-ene;
RT room temperature (approximately 17 to 25° C.);
tR retention time;
m/z mass/charge ratio.

The chemical names were generated by software which used the Lexichem Toolkit (v. 1.40) from OpenEye Scientific Software (www.eyesopen.com) to generate IUPAC conforming names.

### EXAMPLE 1

### N-[2-(Hydroxymethyl)-4-[4-(methylsulfonylmethyl)-6-morpholin-4-yl-pyrimidin-2-yl]phenyl]methanesulfonamide

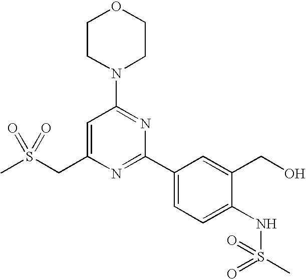

A mixture of N-[4-bromo-2-(hydroxymethyl)phenyl]methanesulfonamide (250 mg), to potassium acetate (263 mg) and bis(pinacolato)diboron (273 mg) in 1,4-dioxane (10 mL) was degassed for 5 minutes. 1,1′-Bis(diphenylphosphino)ferrocenedichloropalladium(II) dichloromethane adduct (44 mg) was added and the reaction was heated to 80° C. for 3 hours. 2-Chloro-4-(methylsulfonylmethyl)-6-morpholin-4-yl-pyrimidine (261 mg), ethanol (0.75 mL), a 2M solution of sodium carbonate (2.7 mL) and additional 1,1′-bis(diphenylphosphino)ferrocenedichloropalladium(II) dichloromethane adduct (54 mg) were added and the heating was continued for a further 3 hours. The cooled reaction mixture was concentrated in vacuo, dissolved in methanol and loaded onto a SCX-2 column. The column was washed with methanol and the compound removed with 7N ammonia in methanol. The solution was concentrated in vacuo and the residue chromatographed on silica, eluting with 0-5% methanol in DCM. The resulting solid was triturated with diethyl ether to give the desired material as a white solid (62 mg).

NMR Spectrum: ¹H NMR (DMSO-d₆) δ3.07 (3H, s), 3.22 (3H, s), 3.74 (5H, s), 4.53 (2H, s), 4.69 (2H, s), 5.44 (1H, s), 6.89 (1H, s), 7.45 (1H, d), 8.23-8.26 (1H, m), 8.43 (1H, s), 9.10 (1H, s)

Mass Spectrum; M+H⁺ 457.

Test (a) average IC₅₀ 5 μM.

The preparation of N-[4-bromo-2-(hydroxymethyl)phenyl]methanesulfonamide is described below:

### N-[4-bromo-2-(hydroxymethyl)phenyl]methanesulfonamide

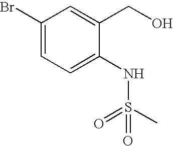

Methyl 5-bromo-2-methanesulfonamido-benzoate (1.34 g) was dissolved in THF (30 mL) and cooled to 0° C. Lithium aluminium hydride (8.7 mL, 1M solution THF) was added slowly to the solution over 15 minutes. The reaction was allowed to warm to room temperature and stir for 2 hours before being quenched with water and filtered. The solution was concentrated in vacuo and the residue chromatographed on silica, eluting with 2.5% methanol in DCM, to give the desired material as a white solid (333 mg).

NMR Spectrum: ¹H NMR (DMSO-d₆) δ3.01 (3H, s), 4.61 (2H, s), 5.40 (1H, s), 7.26 (1H, d), 7.45-7.48 (1H, m), 7.62 (1H, d), 9.05-9.05 (1H, m)

Mass Spectrum; M−H⁺ 280.

### Methyl 5-bromo-2-methanesulfonamido-benzoate

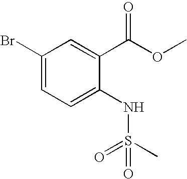

Sodium borohydride (659 mg) was added portion wise to a stirred solution of methyl 2-(bis(methylsulfonyl)amino)-5-bromo-benzoate (1.68 g) in methanol (40 mL) and water (4 mL) at 0° C. The reaction was allowed to warm to room temperature and left to stir for 2 hours. Other portion of sodium borohydride was added at 0° C. and was left to stir for 18 h. Water (10 mL) and Sodium carbonate (2M) (5 mL) was added to quench the reaction. The reaction was vacuumed to dryness and partitioned between ethyl acetate (50 mL) and water (50 mL). Organics dried over magnesium sulphate, filtered and vacuumed to dryness to give the desired material as a white solid (1.34 g).

NMR Spectrum: ¹H NMR (DMSO-d₆) δ3.14 (3H, d), 3.89 (3H, s), 7.52-7.57 (1H, m), 7.78-7.81 (1H, m), 7.99 (1H, d)

Mass Spectrum; M−H⁺ 306.

### Methyl 2-(bis(methylsulfonyl)amino)-5-bromo-benzoate

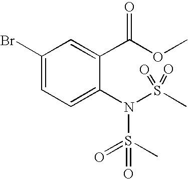

Methyl-2-amino-5-bromobenzoate (1 g) was dissolved in THF (20 mL) and triethylamine (3.6 mL). The mixture was cooled to 0° C. in an ice bath and methanesulfonyl chloride (1 mL) was added slowly. The reaction was allowed to stir at 0° C. for 15 minutes then left to stir for 19 hours at room temperature. The reaction was quenched with water (10 mL) and concentrated in vacuo. The reaction was partitioned between ethyl acetate (50 mL) and water (50 mL), the organics dried (MgSO₄), filtered, and concentrated in vacuo to give the desired material (1.68 g) as a white solid.

NMR Spectrum: ¹H NMR (DMSO-d₆) δ3.52 (6H, s), 3.87 (3H, s), 7.58 (1H, d), 7.94-7.97 (1H, m), 8.10 (1H, d)

Mass Spectrum; M+H⁺ 386.

The preparation of 2-Chloro-4-(methylsulfonylmethyl)-6-morpholin-4-yl-pyrimidine is described below.

### 2-Chloro-4-(methylsulfonylmethyl)-6-morpholin-4-yl-pyrimidine

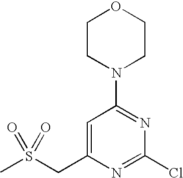

A suspension of 2,4-dichloro-6-(methylsulfonylmethyl)pyrimidine (10.56 g) in DCM (230 mL) was stirred magnetically (under nitrogen) and cooled to −5° C. Triethylamine (6.78 mL) was added followed by the dropwise addition of a solution of morpholine (3.85 mL) in DCM (30 mL) maintaining the reaction temperature below −5° C. The reaction was stirred at room temperature for 1 hour and then the organic mixture washed with water (300 mL). The organic phase was dried (MgSO4), filtered and evaporated to a brown solid which was chromatographed on silica, eluting with 50% ethyl acetate in DCM, to give the desired material (6.81 g) as a white solid.

NMR Spectrum: ¹H NMR (DMSO-d₆) δ3.12 (3H, s), 3.63 (4H, s), 3.68-3.70 (4H, m), 4.45 (2H, s), 6.96 (1H, s)

Mass Spectrum: MH+292.

### 2,4-Dichloro-6-(methylsulfonylmethyl)pyrimidine

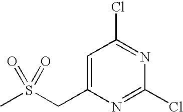

6-(Methylsulfonylmethyl)-1H-pyrimidine-2,4-dione (12.72 g) was suspended in phosphorus oxychloride (125 mL) and heated at reflux under nitrogen for 14 hours. The solution was cooled and concentrated in vacuo to. Iced water (250 mL) was slowly added to the residue and the product then extracted with DCM (3×200 mL). The organics were is concentrated in vacuo to give the desired material as a brown solid (10.56 g).

NMR Spectrum: ¹H NMR (DMSO-d₆) δ3.14 (3H, s), 4.79 (2H, s), 7.88 (1H, s)

Mass Spectrum: (M−H)⁻ 239.

### 6-(Methylsulfonylmethyl)-1H-pyrimidine-2,4-dione

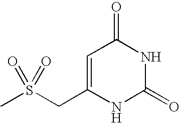

6-(Chloromethyl)uracil (10.00 g) was dissolved in DMF (300 mL) and methanesulphinic acid sodium salt (7.64 g) added. The reaction was heated at 125° C. for 1 hour. The reaction was allowed to cool, filtered and the filtrate concentrated in vacuo to give the desired material as a yellow solid (12.72 g).

NMR Spectrum: ¹H NMR (DMSO-d₆) δ3.10 (3H, s), 4.27 (2H, s), 5.63 (1H, s), 10.94 (1H, s), 11.16 (1H, s).

### EXAMPLE 2

### N-[[4-[4-(Methylsulfonylmethyl)-6-morpholin-4-yl-pyrimidin-2-yl]phenyl]methyl]methanesulfonamide

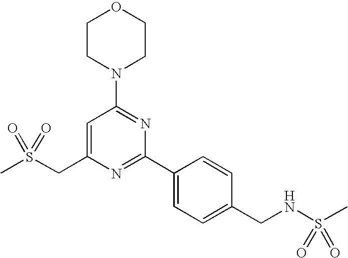

[4-[4-(Methylsulfonylmethyl)-6-morpholin-4-yl-pyrimidin-2-yl]phenyl]methanamine (72 mg) was suspended in DCM (4 mL) and treated with triethylamine (0.056 mL). Methane sulfonyl chloride (0.024 mL) was then added dropwise and the reaction stirred overnight at RT. The reaction was quenched by the addition of water (2 mL) and the layers were separated. The aqueous layer was extracted with DCM (2×2 mL) and the combined organics were dried, filtered and evaporated to a green oil which was purified on the basic prep HPLC system. The product containing fractions were loaded onto a methanol pre-eluted SCX column, washed with methanol, then eluted with 7N ammonia in methanol. The desired fractions were evaporated in vacuo, to give the desired material as a white solid (20 mg).

LCMS Spectrum: MH+ 441.58, Retention Time 1.41 Method: Monitor Acid

NMR Spectrum: ¹H NMR (300.132 MHz, DMSO-d₆) δ2.89 (s, 3H), 3.20 (s, 3H), 3.71 (s, 8H), 4.25 (d, 2H), 4.51 (s, 2H), 6.91 (s, 1H), 7.45 (m, 2H), 7.62 (m, 1H), 8.34 (m, 2H). Test (a) average IC₅₀ 6.8 μM.

The starting material [4-[4-(methylsulfonylmethyl)-6-morpholin-4-yl-pyrimidin-2-yl]phenyl]methanamine was prepared as follows.

[4-[4-(Methylsulfonylmethyl)-6-morpholin-4-yl-pyrimidin-2-yl]phenyl]methanamine

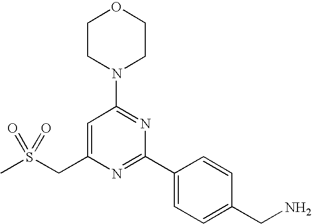

2-Methylsulfanyl-4-(methylsulfonylmethyl)-6-morpholin-4-yl-pyrimidine (228 mg), [4-(aminomethyl)phenyl]boronic acid (310 mg), Copper(I)thiophene-2-carboxylate (373 mg) and Pd(PPh₃)₄ (35 mg) were added to a microwave vessel and 1,4-Dioxane (5 mL) was then added. The system was sealed and heated in a microwave reactor at 130° C. for 1 hour. Further Pd(PPh₃)₄ (10 mg) was added and the reaction heated to 130° C. for 20 minutes. The mixture was transferred to an SCX column, previously eluted with methanol. The SCX column was then flushed with methanol before the desired material was eluted with 7N ammonia in methanol. The fractions were evaporated to give the desired compound as a cream solid (129 mg).

LCMS Spectrum: MH+ 363.55, Retention Time 0.83 Method: Monitor acid

### 2-Methylsulfanyl-4-(methylsulfonylmethyl)-6-morpholin-4-yl-pyrimidine

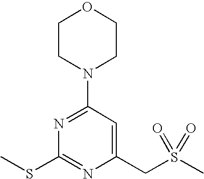

2-Methylsulfanyl-6-(methylsulfonylmethyl)pyrimidin-4-ol (15 g, 63.97 mmol) was heated at reflux in phosphorous oxychloride (100 ml) for approximately 1 hour. Phosphorous oxychloride was evaporated and the residue was neutralised with sodium hydroxide solution and extracted into ethyl acetate. The resultant mixture was then dried over magnesium sulfate, filtered and evaporated to dryness to afford the crude chloro product. This was then dissolved in DCM, morpholine (319 mmol, 28 ml) was added and the reaction stirred at room temperature. Upon completion the resulting precipitate was collected as a white solid. Concentration of the filtrate afforded more solid, giving a combined yield of 13.7 g.

NMR Spectrum: ¹H NMR (300.132 MHz, DMSO) 82.45 (s, 3H), 3.49-3.74 (m, 8H), 4.37 (s, 2H), 6.66 (s, 1H) ppm.

LCMS Spectrum: MH+ 304.50, Retention Time 1.49 min, Method: Monitor Basic

### 2-Methylsulfanyl-6-(methylsulfonylmethyl)pyrimidin-4-ol

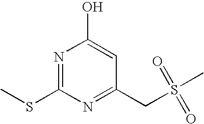

6-(Chloromethyl)-2-methylsulfanyl-pyrimidin-4-ol (19.07 g, 100 mmol) was suspended in acetonitrile (400 ml). To this stirring suspension was added methanesulphinic acid sodium salt (12.255 g, 120 mmol) and DMF (100 ml). The reaction was then heated to 100° C. to to give a dark suspension and monitored by LCMS. Once complete, the solvents were removed and the resultant product added to 1:1 MeOH:DCM (200 ml) and acidified with acetic acid (10 ml). The resultant precipitate was collected, washed with water (200 ml) and MeOH (100 ml) and dried overnight in vacuo to afford the title compound as a white solid, 16.45 g.

NMR Spectrum: ¹H NMR (300.132 MHz, DMSO) 82.50 (s, 3H), 3.12 (s, 3H), 4.39 (s, 2H), 6.25 (s, 1H), 13.09 (s, 1H) ppm.

LCMS Spectrum: MH+ 235.2, Retention Time 0.5 minutes, Method: 5 min Early Base

### 6-(Chloromethyl)-2-methylsulfanyl-pyrimidin-4-ol

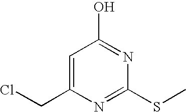

S-Methyl-2-thiopseudourea sulphate (20 g, 71.85 mmol), ethyl 4-chloroacetoacetate (10.755 ml, 79.04 mmol) and sodium carbonate (13.925 g, 107.78 mmol) were dissolved in water (100 ml) and stirred at room temperature overnight. The reaction was monitored by TLC, and once complete, the reaction precipitate was collected and the supernatant was neutralised with 6N hydrochloric acid to yield more reaction precipitate which was also collected. The accumulated precipitate was then washed with water (×3) and an off-white solid was obtained. This was dried in vacuo at 60° C. for 48 hours to yield the desired compound as a pale yellow/white solid, 43.2 g.

NMR Spectrum: ¹H NMR (300.132 MHz, CDCl₃) δ2.59 (s, 3H), 4.35 (s, 2H), 6.41 (s, 1H), 12.70 (s, 1H) ppm

Mass Spectrum: M^{.+} 190

### EXAMPLE 3

### N-[4-[4-(Methylsulfonylmethyl)-6-morpholin-4-yl-pyrimidin-2-yl]phenyl]cyclopropanesulfonamide

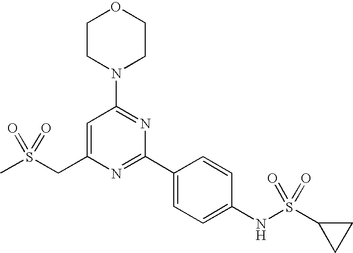

To 4-[4-(methylsulfonylmethyl)-6-morpholin-4-yl-pyrimidin-2-yl]aniline (52 mg) in pyridine (4 mL) was added cyclopropane sulphonyl chloride (53 mg) and the reaction was stirred at RT overnight. To the reaction mixture was then added PS-trisamine resin (200 mg) and the mixture was shaken for 2 hours, then filtered and washed with methanol. The combined organics were evaporated to dryness and then dissolved in DMSO (1 mL) and purified by prep HPLC using a Phenomenex ‘Gemini’ preparative reversed-phase column (5 microns silica, 21.2 mm diameter, 100 nm length) using decreasingly polar mixtures of water and acetonitrile (containing 2% formic acid) as eluent, to yield the title compound. (33 mg).

LCMS Spectrum: M+H⁺ 453.45; Retention Time 2.59 Method: Monitor Acid Test (a) average IC₅₀ 0.88 μM.

The following compounds were prepared in an analogous manner to using the appropriate sulphonyl chloride.

| | | | LCMS | Retention |
|---|---|---|---|---|
| Example | Structure | NAME | MH+ | Time (min) |
| 3a | 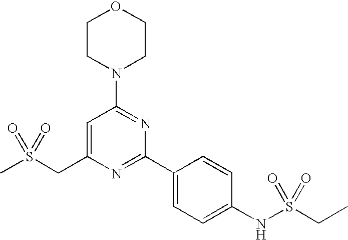 | N-[4-[4- (methylsulfonylmethyl)-6- morpholin-4-yl-pyrimidin-2- yl]phenyl]ethanesulfonamide | 441.43 | 2.53 |
| 3b | 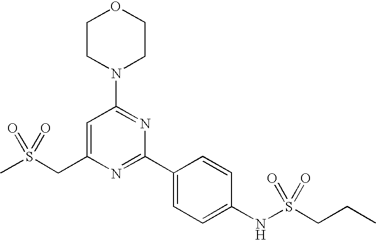 | N-[4-[4- (methylsulfonylmethyl)-6- morpholin-4-yl-pyrimidin-2- yl]phenyl]propane-1- sulfonamide | 455.45 | 2.68 |
| 3c | 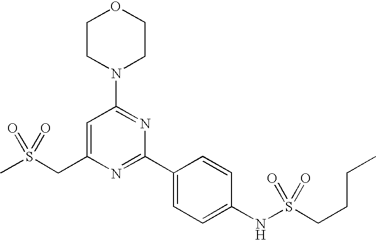 | N-[4-[4- (methylsulfonylmethyl)-6- morpholin-4-yl-pyrimidin-2- yl]phenyl]butane-1- sulfonamide | 469.47 | 2.85 |
| 3d | 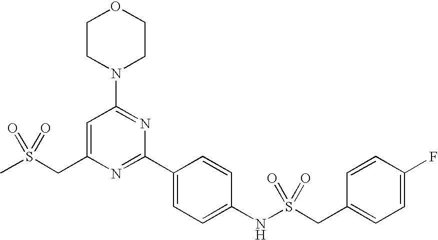 | 1-(4-fluorophenyl)-N-[4-[4- (methylsulfonylmethyl)-6- morpholin-4-yl-pyrimidin-2- yl]phenyl]methanesulfonamide | 521.47 | 2.94 |

Example 3a: Test (a) average IC₅₀ 1.4 μM.
Example 3b: Test (a) average IC₅₀ 4 μM.
Example 3c: Test (a) average IC₅₀ 6 μM.
Example 3d: Test (a) average IC₅₀ 7.1 μM.

The preparation of 4-[4-(methylsulfonylmethyl)-6-morpholin-4-yl-pyrimidin-2-yl]aniline is described below:

### 4-[4-(Methylsulfonylmethyl)-6-morpholin-4-yl-pyrimidin-2-yl]aniline

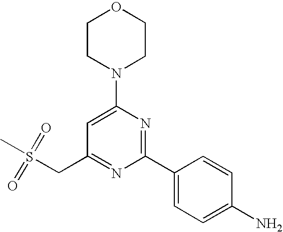

2-Methylsulfanyl-4-(methylsulfonylmethyl)-6-morpholin-4-yl-pyrimidine (1.00 g, 3.3 mmol), 4-aminophenylboronic acid (904 mg, 6.60 mmol), Copper(I)thiophene-2-carboxylate (1.64 g, 8.58 mmol), Pd(PPh₃)₄ (153 mg, 0.04 equiv., 0.13 mmol) were added to a microwave vessel and 1,4-Dioxane (20 mL) added. The system was degassed with N₂, sealed and heated in a microwave reactor at 130° C. for 1 hour. Upon cooling the reaction was poured into water and the resulting precipitate was collected by filtration and dried under vacuum to afford the title compound as an off-white solid. (988 mg)

LCMS Spectrum: MH+ 349.41, Retention Time 1.43 Method: Monitor Acid

NMR Spectrum: ¹H NMR (300.132 MHz, DMSO-d₆) δ3.20 (3H, s), 3.61-3.83 (8H, m), 4.43 (2H, s), 5.57 (1H, s), 6.60 (2H, d), 6.70 (1H, s), 8.04 (2H, d)

### EXAMPLE 4

### N-[4-[4-(Methylsulfonylmethyl)-6-morpholin-4-yl-pyrimidin-2-yl]phenyl]methanesulfonamide

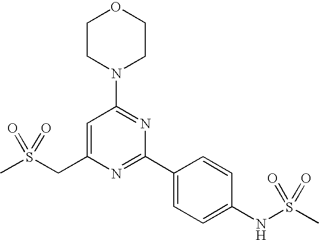

To 4-[4-(methylsulfonylmethyl)-6-morpholin-4-yl-pyrimidin-2-yl]aniline (53 mg, 0.15 mmol)) in pyridine (3 mL) was added methanesulphonyl chloride (21 mg, 0.18 mmol) and the reaction was stirred at RT for 2 hours. Water (10 mL) was added to the mixture and the precipitate collected by filtration. This solid was purified by prep HPLC to yield the desired material as a white solid (29 mg).

LCMS Spectrum: M+H⁺427.4; Retention Time 1.38 Method: Monitor Acid

NMR Spectrum: ¹H NMR (300.132 MHz, DMSO-d₆) δ3.08 (3H, s), 3.22 (3H, s), 3.75 (8H, s), 4.57 (2H, s), 6.93 (1H, s), 7.33 (2H, d), 8.30 (2H, d), 10.12 (1H, s)

Test (a) average IC₅₀ 2.6 μM.

### EXAMPLE 5

### N-[[4-[4-[(3S)-3-Methylmorpholin-4-yl]-6-(methylsulfonylmethyl)pyrimidin-2-yl]phenyl]methyl]methanesulfonamide

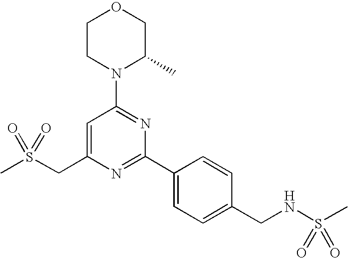

Triethylamine (0.038 mL, 0.27 mmol) was added to a solution of [4-[4-[(3S)-3-methylmorpholin-4-yl]-6-(methylsulfonylmethyl)pyrimidin-2-yl]phenyl]methanamine (100 mg, 0.27 mmol) in DCM (5 mL). Methanesulphonyl chloride (0.021 mL, 0.27 mmol) was added and the reaction stirred at room temperature for 2 hours. The organics were washed with water (5 mL), dried (MgSO₄) and concentrated in vacuo. The crude material was purified on silica, eluting with 0-5% methanol in DCM, to give the desired material as a white solid (90 mg).

LCMS Spectrum: M+H⁺ 455; Retention Time 1.61 Method: Monitor Base

NMR Spectrum: ¹H NMR (400.132 MHz, CDCl₃) δ1.36 (d, 3H), 2.89 (s, 3H), 3.08 (s, 3H), 3.35 (m, 1H), 3.60 (t, 1H), 3.79 (m, 2H), 4.05 (d, 1H), 4.18 (d, 1H), 4.27 (s, 2H), 4.39 (d, 2H), 4.49 (s, 1H), 4.80 (s, 1H), 6.52 (s, 1H), 7.43 (d, 2H), 8.36 (d, 2H). Test (a) average IC₅₀ 4.5 μM.

The preparation of [4-[4-[(3S)-3-methylmorpholin-4-yl]-6-(methylsulfonylmethyl)pyrimidin-2-yl]phenyl]methanamine is described below.

### [4-[4-[(3S)-3-Methylmorpholin-4-yl]-6-(methylsulfonylmethyl)pyrimidin-2-yl]phenyl]methanamine

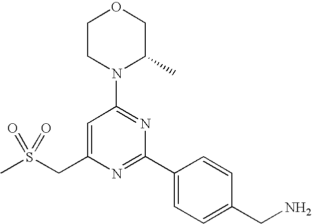

2-Chloro-4-[(3S)-3-methylmorpholin-4-yl]-6-(methylsulfonylmethyl)pyrimidine (1.0 g, 3.27 mmol) was dissolved in a solution of 18% DMF in a mixture of 7:3:2 DME:water:ethanol (9 mL). 4-Aminomethylphenyl boronic acid hydrochloride salt (0.92 g, 4.91 mmol), 2M sodium carbonate solution (3 mL) and dichlorobis(triphenylphosphine) palladium catalyst (115 mg, 0.16 mmol) were then added to the solution and refluxed at 90° C. for 16 hours under nitrogen atmosphere. The reaction was allowed to cool to room temp then partitioned between ethyl acetate and water. The organics were dried over magnesium sulphate, filtered and concentrated to dryness. The crude oil was chromatographed on silica, eluting with 0-5% methanol in DCM, to give the desired material as a cream solid (400 mg).

LCMS Spectrum: M−H⁻ 375; Retention Time 1.58 Method: Monitor Base

NMR Spectrum: ¹H NMR (400.132 MHz, CDCl₃) δ1.36 (d, 3H), 3.08 (s, 3H), 3.34 (m, 1H), 3.60 (m, 1H), 3.75 (m, 1H), 3.83 (d, 1H), 3.94 (s, 2H), 4.05 (m, 1H), 4.18 (m, 1H), 4.26 (s, 2H), 4.49 (m, 1H), 6.50 (s, 1H), 7.40 (d, 2H), 8.33 (d, 2H).

### 2-Chloro-4-[(3S)-3-methylmorpholin-4-yl]-6-(methylsulfonylmethyl)pyrimidine

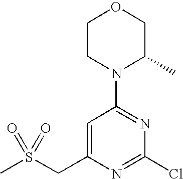

2,4-Dichloro-6-(methylsulfonylmethyl)pyrimidine (30 g, 0.13 mol) was dissolved in dichloromethane and stirred (under nitrogen) at −5° C. Triethylamine (17.4 mL, 0.13 mol) was added to give a clear brown solution. (3S)-3-Methylmorpholine was dissolved in dichloromethane and added dropwise keeping the reaction below −5° C. The cooling bath was then removed and the mixture stirred for 1 hour. The reaction mixture was heated at reflux for 2 hours, then the reaction mixture was washed with water, dried then evaporated. The crude material was purified by preparative HPLC to give the desired material as a solid (19.3 g).

NMR Spectrum: ¹H NMR (400.13 MHz, DMSO-d₆) δ1.21-1.23 (m, 3H), 3.11 (s, 3H), 3.19-3.26 (m, 1H), 3.42-3.49 (m, 1H), 3.58-3.62 (1H, m), 3.73 (d, 1H), 3.92-3.96 (m, 2H), 4.27-4.31 (m, 1H), 4.45 (s, 2H), 6.92 (s, 1H)

LCMS Spectrum: MH+ 306, retention time 1.42 min, Method 5 Min Acid

The preparation of 2,4-dichloro-6-(methylsulfonylmethyl)pyrimidine was described earlier.

### EXAMPLE 6

### N-[4-[4-[(3S)-3-Methylmorpholin-4-yl]-6-(methylsulfonylmethyl)pyrimidin-2-yl]phenyl]methanesulfonamide

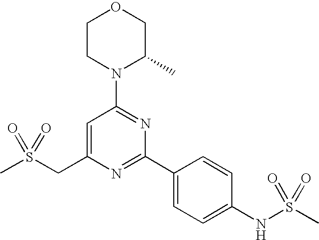

2-Chloro-4-[(3S)-3-methylmorpholin-4-yl]-6-(methylsulfonylmethyl)pyrimidine (150 mg, 0.49 mmol) was dissolved in a solution of 18% DMF in a mixture of 7:3:2 DME:water:ethanol (1.5 mL). 4-Aminomethylphenyl boronic acid hydrochloride salt (0.74 mmol), 2M sodium carbonate solution (0.5 mL) and dichlorobis(triphenylphosphine) palladium catalyst (18 mg, 0.02 mmol) were then added to the solution and refluxed at 90° C. for 16 hours under nitrogen atmosphere. The reaction was allowed to cool to room temp then partitioned between ethyl acetate and water. The organics were dried over magnesium sulphate, filtered and concentrated to dryness. The crude solid was dissolved in DCM, insoluble impurities removed by filtration then the filtrate purified by prep HPLC to give the desired compound as a white solid (87 mg).

NMR Spectrum: ¹H NMR (400.13 MHz, DMSO-d₆) δ 1.24 (3H, d), 2.93 (3H, s), 3.21 (3H, s), 3.24 (1H, m), 3.50 (1H, m), 3.65 (1H, m), 3.78 (1H, d), 3.98 (1H, m), 4.16 (1H, d), 4.43 (1H, s), 4.48 (2H, s), 6.76 (1H, s), 7.17 (2H, d), 8.21 (2H, d)

LCMS Spectrum: M+H⁺ 441; Retention Time 1.45 Method: Monitor Acid Test (a) average IC₅₀ 1 μM.

1. A compound of formula (I) 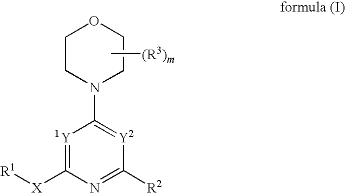
or a pharmaceutically acceptable salt thereof;
wherein
m is 0, 1, 2, 3 or 4;
¹Y and Y² are independently N or CR⁸ provided that one of ¹Y and Y² is N and the other is CR⁸;
X is a linker group selected from —CR⁴═CR⁵—, —CR⁴═CR⁵CR⁶R⁷—, —CR⁶R⁷CR⁵═CR⁴—, —C≡C—, —C—≡CCR⁶R⁷—, —CR⁶R⁷C≡C—, —NR⁴CR⁶R⁷—, —OCR⁶R⁷—, —SCR⁶R⁷—, —S(O)CR⁶R⁷, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
R¹ is a group selected from hydrogen, C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁶, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is substituted by —NR¹⁷SO₂R¹⁸ and optionally substituted by one or more substituent group independently selected from halo, cyano, nitro, —R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹, R¹² and R¹⁷ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, R¹⁶ and R¹⁸ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

2. The compound, or a pharmaceutically acceptable salt thereof, according to claim 1 wherein the compound of formula (I) is a compound of formula (Ia) or (Ib) 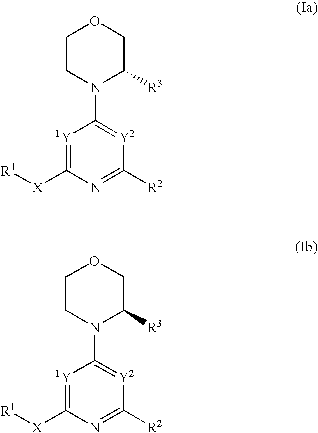
or a pharmaceutically acceptable salt thereof, wherein R¹, R², R³, X, Y¹ and Y² are as defined for the compound of formula (I).

3. The compound, or a pharmaceutically acceptable salt thereof, according to claim 1 wherein R³ is methyl.

4. The compound, or a pharmaceutically acceptable salt thereof, according to claim 1 wherein Y¹ is CR⁸ and Y² is N.

5. The compound, or a pharmaceutically acceptable salt thereof, according to claim 4 wherein Y¹ is CH and Y² is N.

6. The compound, or a pharmaceutically acceptable salt thereof, according to claim 1 wherein X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷, —NR⁴C(O)NR⁵CR⁶R⁷—, —S(O)₂NR⁴CR⁶R⁷, —C(O)NR⁴— and —NR⁴C(O)—.

7. The compound, or a pharmaceutically acceptable salt thereof, according to claim 6 wherein X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴—, and —NR⁴C(O)—.

8. The compound, or a pharmaceutically acceptable salt thereof, according to claim 7 wherein X is a linker group selected from —NR⁴—CH₂—, —OCH₂—, —OCH(CH₃)—, —OC(CH₃)₂—, —SCH₂—, —SCH(CH₃)—, —SC(CH₃)₂—, —S(O)CH₂—, —S(O)CH(CH₃)—, —S(O)C(CH₃)₂—, —S(O)₂CH₂—, —S(O)₂CH(CH₃)—, —S(O)₂C(CH₃)₂—, —C(O)NR⁴— and —NR⁴C(O)—.

9. The compound, or a pharmaceutically acceptable salt thereof, according to claim 8 wherein X is —S(O)₂CH₂—, —S(O)₂CH(CH₃)— or —S(O)₂C(CH₃)₂—.

10. The compound, or a pharmaceutically acceptable salt thereof, according to claim 1 wherein R¹ is a group selected from adamantyl, methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent groups selected from halo, cyano, nitro, R⁹, —OR⁹, —COR⁹, —CONR⁹R¹⁰, —NR⁹R¹⁰ and —NR⁹COR¹⁰.

11. The compound, or a pharmaceutically acceptable salt thereof, according to claim 10 wherein R¹ is a group selected from methyl, ethyl, propyl, butyl, isobutyl, tert-butyl, cyclopropyl, cyclopentyl cyclohexyl, phenyl, benzyl, phenethyl, pyridinyl, pyrazolylethyl, furanylmethyl, thienylmethyl, thiazolylmethyl, thiadiazolylmethyl and pyrazinylethyl, which group is optionally substituted by 1 or 2 substituent groups selected from amino, halo, cyano, methyl, methoxy, trifluoromethyl, trifluoromethoxy, —NHCOCH₃, —CONH₂ and —CONHCH₃.

12. The compound, or a pharmaceutically acceptable salt thereof, according to claim 11 wherein R¹ is a group selected from methyl, isopropyl, cyclopropyl, cyclohexyl, —CH₂CH₂OH, —CH₂CH₂NC(O)CH₃, phenyl, 4-fluorophenyl, 2-chlorophenyl, 2-trifluoromethylphenyl, 2-methoxyphenyl, 2-methylphenyl, 4-acetamidophenyl, 4-aminophenyl, pyridin-4-yl, pyridin-2-yl, 2-oxopyrrolidin-3-yl, thiazol-2-yl, 4-methylthiazol-2-yl, and 3-methyl-1,3,4-thiadiazol-2-yl.

13. The compound, or a pharmaceutically acceptable salt thereof, according to claim 12 wherein R¹ is methyl.

14. The compound, or a pharmaceutically acceptable salt thereof, according to claim 1 wherein R² is a group selected from a 5- or 6-membered carbocyclyl or heterocyclyl which group is substituted by —NR¹⁷SO₂R¹⁸ and optionally substituted by one or more substituent groups independently selected from halo, cyano, nitro, —R¹¹, —OR¹¹, —COR¹¹, —CONR¹¹R¹², —NR¹¹R¹² and —NR¹¹COR¹².

15. The compound, or a pharmaceutically acceptable salt thereof, according to claim 4 wherein R² is a group selected from a 6-membered aryl and a 5- or 6-membered heteroaryl which group is substituted by —NR¹⁷SO₂R¹⁸ and optionally substituted by one or more substituent groups independently selected from halo, cyano, nitro, —R¹¹, —OR¹¹, —COR¹¹, —CONR¹¹R¹², —NR¹¹R¹² and —NR¹¹COR¹².

16. The compound, or a pharmaceutically acceptable salt thereof, according to claim 15 wherein R² is a group selected from phenyl, pyrrolyl, imidazolyl, pyrazolyl, furanyl, thienyl, pyridinyl, pyrimidinyl, pyridazinyl, and thiazolyl which group is substituted by —NR¹⁷SO₂R¹⁸ and optionally substituted by one or more substituent groups independently selected from halo, cyano, nitro, —R¹¹, —OR¹¹, —COR¹¹, —CONR¹¹R¹², —NR¹¹R¹² and —NR¹¹COR¹².

17. The compound, or a pharmaceutically acceptable salt thereof, according to claim 16 wherein R² is a group selected from phenyl, pyrrolyl, imidazolyl, pyrazolyl, furanyl, thienyl, pyridinyl, pyrimidinyl, pyridazinyl, and thiazolyl which group is substituted by —NR¹⁷SO₂R¹⁸ and optionally substituted by one or more substituent group independently selected from fluoro, methyl, methoxy, hydroxymethyl, cyanomethyl, —CONH₂, —CONHCH₃ and —CON(CH₃)₂.

18. The compound, or a pharmaceutically acceptable salt thereof, according to claim 17 wherein R² is phenyl or pyridyl substituted by —NR¹⁷SO₂R¹⁸ and optionally substituted by one or more substituent groups independently selected from fluoro, methyl, methoxy, hydroxymethyl, cyanomethyl, —CONH₂, —CONHCH₃ and —CON(CH₃)₂.

19. The compound, or a pharmaceutically acceptable salt thereof, according to claim 18 wherein R² is 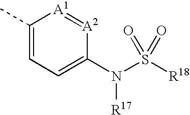
wherein A¹ and A² are selected CH or No provided that at least one of A¹ or A² is CH.

20. The compound, or a pharmaceutically acceptable salt thereof, according to claim 14 wherein R¹⁷ is hydrogen.

21. The compound, or a pharmaceutically acceptable salt thereof, according to claim 1 wherein R¹⁸ is hydrogen or a group selected from C₁₋₆alkyl, C₃₋₆cycloakyl, aryl, heteroaryl, arylC₁₋₆alkyl and heteroarylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl.

22. The compound, or a pharmaceutically acceptable salt thereof, according to claim 21 wherein R¹⁸ is hydrogen or a group selected from methyl, ethyl, propyl, i-propyl, butyl, i-butyl, t-butyl, pentyl, cyclopropyl, cyclobutyl, cyclopentyl, cyclohexyl, phenyl, thienyl, imidazoylmethyl, isoxazolyl, pyrazolyl, pyridinyl and pyrimidinyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₆alkylcarbamoyl and bis(C₁₋₆alkyl)carbamoyl.

23. The compound, or a pharmaceutically acceptable salt thereof, according to claim 22 wherein R¹⁸ is hydrogen or a group selected from methyl, ethyl propyl, i-propyl, butyl, i-butyl, t-butyl, pentyl, cyclopropyl, cyclobutyl, cyclopentyl, cyclohexyl, —CH₂(cyclopropyl), —CH₂CH₂NMe₂, —CH(CH₃)CH₂OH, —C(CH₃)₂CH₂OH, —CH₂CH₂OH, —CH₂CH₂CH₂OH, 4-methylphenyl, 4-chlorophenyl, 4-trifluoromethylphenyl, 4-fluorophenyl, 4-methoxyphenyl, 3,4-difluorophenyl, thien-2-yl, —CH₂(imidazol-2-yl), —CH₂(imidazol-3-yl), isoxazolyl-3-yl, 6-oxo-1H-pryrdin-2-yl, 5-methylisoxazol-3-yl, 1-methylpyrazol-4-yl, 6-methoxypryridin-3-yl, 5-fluoropyridin-2-yl, pyrimidin-2-yl, and 1H-pyrazol-3-yl.

24. The compound, or a pharmaceutically acceptable salt thereof, according to claim 23 wherein R¹⁸ is hydrogen or a group selected from methyl, ethyl, propyl, butyl, cyclopropyl, and 4-fluorophenyl.

25-29. (canceled)

30. A method for producing an anti-proliferative effect in a warm-blooded animal, in need of such treatment which comprises administering to the animal an effective amount of a compound of formula (I), or a pharmaceutically acceptable salt thereof, according to claim 1.

31. A method for treating cancer, inflammatory diseases, obstructive airways diseases, immune diseases or cardiovascular diseases in a warm blooded animal in need of such treatment which comprises administering to the animal an effective amount of a compound of formula (I), or a pharmaceutically acceptable salt thereof, according to claim 1.

32. A pharmaceutical composition comprising a compound of formula (I), or a pharmaceutically acceptable salt thereof, according to claim 1 in association with a pharmaceutically acceptable diluent or carrier.

33. (canceled)
